(19) United States
(12) Patent Application Publication
(10) Pub. No.: US 2004/0242523 A1
(43) Pub. Date: Dec. 2, 2004

(54) Chemo-inducible cancer gene therapy

(75) Inventors: Ralph R. Weichselbaum, Chicago, IL (US); Donald W. Kufe, Wellesley, MA (US)

Correspondence Address: FULBRIGHT & JAWORSKI L.L.P., 600 CONGRESS AVE., SUITE 2400, AUSTIN, TX, 78701, US

(73) Assignee: ana-Farber Cancer Institue and The Univiersity of Chicago

(21) Appl. No.: 10/795,090

(22) Filed: Mar. 5, 2004

Related U.S. Application Data
(60) Provisional application No. 60/452,489, filed on Mar. 6, 2003.

(51) Int. Cl. A61K 48/00; A61K 31/704

(52) U.S. Cl. 514/44; 514/34

(57) ABSTRACT

The present invention provides a method of inhibiting a hyperproliferative cell comprising providing to the cell a TNF-α expression construct comprising a chemotherapeutic responsive promoter and a chemotherapeutic selected from doxorubicin, cyclophosphamide, 5-fluorouracil, taxol and gemcitabine.

This application claims benefit of priority from U.S. Provisional Serial No. 60/452,489, filed March 6, 2003, the entire contents of which is hereby incorporated by reference.

### BACKGROUND OF THE INVENTION

1. Field of the Invention

The present invention relates generally to the fields of cancer biology, cancer gene therapy and molecular biology. More particularly, it concerns methods and composition for combination treatment of hyperproliferative diseases.

2. Description of Related Art

The growth of normal cells is a controlled process. A cell whose growth is not controlled may proliferate more frequently, becoming hyperproliferative. Cancer is an example of a disease characterized by hyperproliferative cells.

Diseases or conditions such as hyperproliferative disease would benefit from destruction, alteration, or inactivation of the hyperproliferative cells, or by replacement of a missing or abnormal gene products that result in the hyperproliferation. In certain situations, the hyperproliferative cells are focused in a recognizable tissue. Current methods of therapy that attempt to seek and destroy those tissues, or to deliver necessary gene products to them, have serious limitations. Additionally, there are few effective options for the treatment of many common cancers.

Standard cancer treatment methods, including radiotherapy and chemotherapy, involve damaging the DNA of the cancer cell. The cellular response to normal DNA damage includes activation of DNA repair, cell cycle arrest and lethality (Hall, 1988). For example, the induction of DNA double-strand breaks results in lethal chromosomal aberrations that include deletions, dicentrics, rings, and anaphase bridges (Hall, 1994).

Another approach to treating cancers is gene therapy. This involves the transfer of a foreign gene into a cancer cell, for example a tumor suppressor or inducer of apoptosis, under conditions suitable for expression of the gene. Once expressed, the gene product confers a beneficial effect on the tumor cell by either slowing its growth, inhibiting its metastatic potential, or killing it outright. However, the clinical effectiveness of cancer gene therapy has been limited by 1) lack of control of therapeutic gene expression within the tumor, and 2) selective targeting of the vector to the tumor. Several strategies have been proposed for the control of gene expression. One strategy is transcriptional targeting in which the promoter regulating the therapeutic gene is activated by tumor-selective transcription factors. Examples include the use of the MUC-1 promoter in breast cancer and the CEA promoter in colon cancer (Kurihara et al., 2000; Konishi et al., 1999).

Combining one or more of these methods is a powerful tool given the heterogeneity of many tumors, and the fact that mono-therapies are far less effective than combinations. However, radio-, chemo- and gene therapy all have the potential for toxic effects. Thus, being able to reduce toxicity, for example, by reducing the amount of radiation/drug/vector administered, is highly advantageous. For example, tumor necrosis factor-alpha (TNF-α), which has antitumor properties, has been studied as a systemic gene therapy treatment for cancer in phase 1 studies, but toxicity has limited the therapeutic index of this cytokine (Spriggs et al., 1988; Demetri et al., 1989). Also, combinations of systemic TNF-α and chemotherapy have been investigated in a few clinical trials with limited success (Nakamoto et al., 2000).

These anti-cancer agents play a role in the production of oxygen and other free radical species that lead to DNA damage, peroxidation of lipids, protein modification and cellular death (Kubota, 1991; Smeta, 1994). Agents other than IR that increase intracellular ROIs (Houben, 1971) include the widely used anti-cancer drugs doxorubicin (Doroshow, 1986), cisplatin (Sodhi and Gupta, 1986; Senturker et al., 2002), cyclophosphamide (Sulkowska et al., 1998), 5-fluorouracil (Ueta et al., 1999), gemcitabine (van der Donk et al., 1998), and paclitaxel (Varbiro et al., 2001). Previous studies showed that IR activates the transcription of the Egr-1 CArG sequences by production of ROIs (Datta et al., 1993; (Nose et al., 1991).

On the other hand, chemotherapeutic agents such as cisplatin and other platinum analogues are currently employed in the treatment of several cancers including head and neck, esophageal, lung, testis, ovarian, and bladder cancers. Additionally, cisplatin is used concurrently with irradiation (IR) as a radiosensitizer. In spite of the relative efficacy of cisplatin, tumor-resistance has limited the role of cisplatin in curative cancer chemotherapy (Johnson and Stevenson, 2001). Tumor-derived mechanisms of cisplatin-resistance include an increase in DNA repair of cisplatin adducts in tumor cells, an increase in glutathione, which inhibits free-radical formation and subsequent DNA damage, and a relative decrease in uptake of cisplatin by resistant cells (Kartalou and Essigmann, 2001). The combination of cisplatin with other chemotherapeutic agents, especially 5-FU and VP-16, has increased the therapeutic index of both agents in some human tumors (Kucuk et al., 2000), but other strategies are needed to increase the efficacy of cisplatin.

Thus, there remains a continued need for effective cancer therapies. Combination therapy is benefical in that it allows for lower dosages and decreases toxicity of the single agent or both agents, but the full potential of combination therapy has yet to be realized in clinical oncology.

### SUMMARY OF THE INVENTION

The present invention overcomes the deficiencies in the art and provides methods that enhance the efficacy of a gene therapy-chemotherapy combination in the treatment of cancers.

Thus, in accordance with the present invention, there is provided a method of inhibiting a hyperproliferative cell comprising contacting the hyperproliferative cell with (a) an expression construct comprising a chemotherapeutic responsive promoter operably linked to a nucleic acid encoding a therapeutic gene, and (b) a chemotherapeutic agent selected from doxorubicin, cyclophosphamide, 5-fluorouracil, taxol or gemcitabine.

The chemotherapeutic responsive promoter of the present invention may be an Egr-1 promoter, a c-jun promoter or a c-fos promoter. In particular embodiments, the chemotherapeutic responsive promoter may be operatively linked to a nucleic acid encoding a tumor therapeutic gene such as TNF-α.

In further embodiments of the invention, a hyperproliferative cell may be contacted with chemotherapeutic agents such as doxorubicin, cyclophosphamide, 5-fluorouracil, taxol or gemcitabine. In some embodiments of the invention, the hyperproliferative cell may be contacted with two or more of doxorubicin, cyclophosphamide, 5-fluorouracil, taxol or gemcitabine.

In particular embodiments of the present invention, the hyperproliferative cell may be a cancer cell, a metastatic cancer cell or a multi-drug resistant cancer cell. In further embodiments, the cancer cell may be a breast cancer cell, an ovarian cancer cell, a pancreatic cancer cell, a prostate cancer cell, a colon cancer cell, a bladder cancer cell, a lung cancer cell, a liver cancer cell, a stomach cancer cell, a testicular cancer cell, an uterine cancer cell, a brain cancer cell, a lymphatic cancer cell, a skin cancer cell, a bone cancer cell, a kidney cancer cell, a rectal cancer cell, or a sarcoma. In some embodiments, the hyperproliferative cell may be a recurrent cancer cell. In still further embodiments, the hyperprolifeative cell is located in a mammal such as a human.

In further embodiments, the hyperproliferative cell contacted with an expression construct such as a viral vector expression construct or a non-viral vector expression construct. The viral vector of the present invention may be an adenoviral vector, adeno-associated viral vector, retroviral vector, lentiviral vector, herpesviral vector, papilloma viral vector, or hepatitis B viral vector. In a further embodiment of the invention, the non-viral vector may be comprised in a liposome.

As described herein, inhibiting comprises induction of apoptosis, cancer cell killing, inhibition of metastasis, induction of tumor regression, reduction of tumor burden, a decrease in tumor cell growth, or suppression of tumor cell growth. Such inhibition may occur in a cell or in a subject having a hyperproliferative disease. Thus, the present invention provides a method of inhibiting metastasis of a cancer cell, or killing a cancer cell, or inducing apoptosis in a cancer cell. In further embodiments of the invention, inhibiting comprises reducing tumor burden or inducing tumor regression in a mammal such as a human.

In some embodiments, the tumor therapeutic gene or chemotherapeutic agent may be administered more than once. In further embodiments, the tumor therapeutic gene may be administered intratumorally, intramuscullarly, intravenously or intraaterially. In still further embodiments, the chemotherapuetic agent may be administered intratumorally, intramuscularly, intravenously or intraaterially. In particular embodiments of the invention, the tumor therapeutic gene may be delivered to a cell, before, at the same time as, or after the chemotherapeutic agent.

In particular embodiments, the invention comprises providing the mammal an adjunct cancer therapy with the therapeutic gene and chemotherapeutic agent of the invention. Such adjunct cancer therapy may be a second chemotherapy, a radiotherapy, an immunotherapy, a hormonal therapy, or a gene therapy.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of“one or more,” “at least one,” and “one or more than one.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Ad.Egr.TNF.

FIG. 2. Mechanism of action of Ad.Egr.TNF.

FIG. 3. Chemo-induction in PC-3 cells in vitro.

FIG. 4. Chemo-induction in PROb cells in vitro.

FIG. 5. Chemo-induction in PC-3 xenografts.

FIG. 6. Chemo-induction in PROb xenografts.

FIG. 7. Fractional tumor volumes in PC-3 xenografts treated with normal saline (·),doxorubicin alone (∘), Ad.Egr.TNF alone (●) or combination of Ad.Egr.TNF plus doxorubicin (×).

FIGS. 8A-8B. Induction of TNF-α protein. TNF-α production by Ad.Egr-TNF.11D-infected cells exposed to cisplatin (250 μM), doxorubicin, (3 μM), 5-FU (100 mM), gemcitabine (3 mM) or paclitaxel (14 μM) for 24 h was measured by ELISA. FIG. 8A—Significant increases in levels of TNF-α protein were detected in PC-3 cells following exposure to Ad.Egr-TNF.11D plus cisplatin (53.2 pg/ml; p<0.001), 5-FU (943.7 pg/ml; p<0.001), gemcitabine (38.3 pg/ml; p<0.001) and paclitaxel (23.8 pg/ml; p<0.001) compared with exposure to Ad.Egr-TNF.11D alone (14 pg/ml). Doxorubicin was toxic to PC-3 cells. FIG. 8B—PROb cells infected with Ad.Egr-TNF.11D produced 130 pg/ml of TNF-α protein. The combination of Ad.Egr-TNF.11D and chemotherapeutic agents significantly increased TNF-α levels; cisplatin (163.3 pg/ml; p<0.04), doxorubicin (961.9 pg/ml; p<0.001), 5-FU (215.9 pg/ml; p=0.02), gemcitabine (460 pg/ml; p<0.001), and paclitaxel (583.2 pg/ml; p<0.001).

FIGS. 9A-9B. The effect of NAC on the induction of TNF-A protein by PC-3 cells. FIG. 9A—In the presence of increasing concentrations of NAC (from 10 mM to 30 mM), TNF-α production by PC-3 cells infected with Ad.Egr-TNF.11D and treated with 5-FU (0 and 100 mM) falls below constitutive levels from PC-3 cells infected with Ad.Egr-TNF.11D alone. Data are reported as mean ± SEM. FIG. 9B—The effect of NAC on the chemo-induction of TNF-α protein TNF-α production by Ad.Egr-TNF.11D-infected cells exposed to cisplatin (250 μM), doxorubicin, (3 μM), 5-FU (100 mM), gemcitabine (3 mM) or paclitaxel (14 μM) with or without addition of 200 mM N-acetyl cysteine (NAC) was measured by ELISA. In PC-3 cells (FIG. 9A) and PROb cells (FIG. 9B) the addition of NAC significantly reduced the TNF-α levels induced by the panel of chemotherapeutic agents tested.

FIGS. 10A-10B. In vivo measurement of TNF-α protein. FIG. 10A—PC-3 xenografts. A significant increase in TNF-α protein concentration was observed following treatment with Ad.Egr-TNF.11D and cisplatin (1150.91±361.35 pg/mg protein, p=0.062), cyclophosphamide (1661.83±343.12 pg/mg protein, p<0.001), doxorubicin (1577.27±284.35 pg/mg protein, p<0.001), 5-FU (1653.33±362.70 pg/mg protein, p<0.001) and gemcitabine (1169.09±195.47 pg/mg protein, p<0.001) compared with Ad.Egr-TNF.11D treatment alone (376.33±64.22 pg/mg protein). FIG. 10B—Significant induction of TNF-α was also detected in PROb tumors following combined treatment with Ad.Egr-TNF.11D and chemotherapy including cisplatin (6912.50±1013.73 pg/mg protein, p=0.002), cyclophosphamide (7923.53±1362.56 pg/mg protein, p<0.001), doxorubicin (6229.41±1137.10 pg/mg protein, p<0.001, 5-FU (5094.12±923.81 pg/mg protein, p=0.023), and gemcitabine (6723.53±1173.06 pg/mg protein, p<0.001) compared with Ad.Egr-TNF.11D alone (2688.24±533.57 pg/mg protein). Data are reported as the mean±SEM.

FIGS. 11A-11B. Xenograft regrowth studies. FIG. 11A—In PC-3 xenografts, combined treatment with Ad.Egr-TNF.11D and doxorubicin produced significant tumor regression compared with Ad.Egr-TNF.11D alone on days 16 (p=0.025), 20 (p=0.039) and 23 (p=0.006). FIG. 11B—In PROb xenografts, significant tumor regression was observed in the tumors receiving combined treatment with Ad.Egr-TNF.11 D and doxorubicin compared with Ad.Egr-TNF.11D alone on days 23 (p=0.027) and 27 (p=0.015). Day 0 represents the first day of treatment. Data are reported as mean ± SEM.

### DESCRIPTION OF ILLUSTRATIVE EMBODIMENTS

A. The Present Invention

The present invention provides a method for inhibiting a hyperproliferative cell. Such methods comprise contacting a hyperproliferative cell with an expression construct encoding a tumor therapeutic gene, such as TNF-α, in combination with a chemotherapeutic such as doxorubicin, cyclophosphamide, 5-FU, taxol or gemcitabine together as a therapeutic modality for treating cancers. The present invention further employs the use of adjunct therapies with the combined tumor therapeutic gene and chemotherapeutic composition for inhibiting, reducing, suppressing or ameliorating hyperproliferative growth in a subject.

More particularly, an expression construct encoding a tumor therapeutic gene was engineered by ligating the CArG (CC(A/T)₆GG) elements of the Egr-1 gene promoter upstream to a cDNA encoding human tumor necrosis factor-a (TNF-α) thereby, creating a replication defective adenoviral vector, Ad.Egr-TNF.11D. The inventors report here that Ad.Egr-TNF.11D is activated by the clinically important anti-cancer agents cisplatin, cyclophosphamide, doxorubicin, 5-flourouracil (5-FU), gemcitabine and paclitaxel. N-acetylcysteine (NAC), a free radical scavenger, blocked induction of TNF-α by anti-cancer agents, supporting a role for reactive oxygen intermediates (ROIs) in activation of the CArG sequences. Importantly, resistance of PC-3 human prostate carcinoma and PROb rat colon carcinoma tumors to doxorubicin in vivo was reversed by combining doxorubicin with Ad.Egr-TNF and resulted in significant anti-tumor effects. Treatment with Ad.Egr-TNF.11D has been associated with inhibition of tumor angiogenesis. In this context, a significant decrease in tumor microvessel density was observed following combined treatment with doxorubicin and Ad.Egr-TNF.11D as compared to either agent alone. These data demonstrate that Ad.Egr-TNF.11D is activated by diverse anti-cancer drugs. Thus, the inventors hypothesized that clinically employed chemotherapeutic agents that increase ROIs could also be employed to activate Ad.Egr-TNF.11D in a chemo-inducible gene therapy strategy.

The inventors report that Ad.Egr-TNF.11D can be activated by anthracyclines, alkylating agents, anti-metabolites and microtubule stabilizing agents through the production of ROIs. Importantly, combined treatment with doxorubicin and Ad.Egr-TNF.11D produces greater anti-tumor effects than either agent alone in tumor models which are resistant to doxorubicin. These anti-tumor effects were achieved by selective induction of Ad.Egr-TNF.11D within the tumor volume, inhibition of tumor angiogenesis and/or direct cytotoxic effects mediated by the combination of Ad.Egr-TNF.11D and doxorubicin. Thus, the present invention provides a chemo-inducible gene therapy to overcome tumor resistance to broad classes of cancer chemotherapeutic agents.

B. Ad.Egr-TNF.11D

In a particular embodiment, the present invention employs a replication objective adenoviral vector Ad.Eg.TNV.11D in a chemo-inducible gene therapy for the treatment of hyperproliferative diseases or conditions. In a transcriptional targeting strategy to localize TNF-α induction to the tumor bed, ionizing radiation (IR) was employed to activate the radio-inducible CArG sequences of the Egr-1 promoter ligated upstream of a cDNA encoding the human TNF-α gene. For delivery, the Egr-TNF construct was integrated into a replication defective adenovirus (E1-, partially E3-deleted) to construct the Ad.Egr-TNF vector (Hallahan et al., 1995). Preclinical experiments demonstrated synergistic anti-tumor effects following combined treatment with Ad.Egr-TNF and IR in human head and neck, prostate, esophageal, and glioma xenografts, (Hallahan et al., 1995; Mauceri et al., 1997; Chung et al., 1998; Gupta et al., 2002; Staba et al., 1998). TNF-60 production was confined to the tumor bed and no systemic toxicity was detected. Histopathological analyses demonstrated damage to the tumor microvasculature, but not to adjacent normal tissues (Mauceri et al., 1996).

Ad.Egr-TNF.11D has been studied in two separate phase I clinical trials with radiation therapy (Mundt et al., 2002; Sharma et al., 2001a; Sharma et al., 2001b; Hanna et al., 2002). The first trial included patients with tumors of different histological types who required palliative radiotherapy. Tumors were directly injected with the Ad.Egr-TNF.11D vector at concentrations of vector ranging from 4×10⁷-4×10¹¹ particle units (p.u). The doses of radiation ranged from 30-66.6 Gy. 70% of the patients (21/30) demonstrated a tumor response or tumor stabilization which was noted mostly at the higher dose levels (4×10⁹-4×10¹¹ p.u.) of the vector. There were 5 complete responses (CR), which included 3 patients with melanoma a typically radioresistant histological tumor and one patient with rectal cancer and another with breast cancer (Senzer et al., 2004). In the second phase 1 trial, patients with large unresectable soft tissue sarcomas of the extremities were treated with Ad.Egr-TNF.11D (4×10⁹-4×10¹¹ p.u. in 1 log increments) and 50 Gy. Objective responses were observed in 11 of 13 patients (85%). Pathological CRs were noted in 2 patients with very large tumors (328-338 cm2). Eight patients exhibited a partial response (PR). Four patients experienced 95% tumor necrosis, 3 patients 80% necrosis and one patient 60% necrosis (Mundt, et al., submitted). Taken together these findings demonstrate the safety of Ad.Egr-TNF.11D+IR combined treatment (Sharma et al., 2001a; Hanna et al., 2002). Additionally, sterilization of radioresistant and/or very large tumors suggests that Ad.Egr-TNF.11D may enhance radiocurabiltiy in some patients.

C. Tumor Therapeutic Genes

In accordance with the present invention, a tumor therapeutic gene is expressed from an expression construct, driven by a chemotherapeutic promoter. TNF-α is exemplified, but a large number of other possible genes, described below, may be used.

1. TNF-alpha

Tumor necrosis factor-α (TNF-α) is a cytokine produced by a variety of cells including macrophages, lymphocytes and natural killer cells. TNF-α is directly cytotoxic to some tumor cells in vitro, although direct cell killing frequently requires inhibition of protein synthesis with compounds such as cycloheximide (Ruff and Gifford, 1981; Wallach, 1984; Gonen et al., 1992). The anti-tumor activity of TNF-60 is predominantly mediated by destruction of the tumor vasculature (Nawroth and Stern, 1986, Watanabe et al., 1988; Tartaglia et al., 1993; Robaye et al., 1991; Havell et al., 1988; Obrador et al., 2001; Slungaard et al., 1990; Mauceri et al., 2002) and this cytokine was named for its induction of hemorrhagic necrosis in experimental tumors, (Vilcek et al., 1986; Carswell et al., 1975). Based on anti-tumor effects in animal models (Old, 1985; Fiers, 1991), clinical trials were performed using intravenous delivery of TNF-α. The therapeutic utility of TNF-α, however, was limited by serious side effects, which included fatigue, weight loss, nausea, cachexia, and shock (Spriggs et al., 1987; Wiedenmann et al., 1989; Brown et al., 1991; Budd et al., 1991; Mittelman et al., 1992; Hallahan et al., 1995). To decrease systemic toxicity of TNF-α, regional delivery approaches were developed to restrict TNF-60 to the tumor bed in isolated limb and liver perfusions, (Lejeune et al., 1994; Hill et al., 1993; Lienard et al., 1994; Kuppen et al., 1997; Alexander et al., 1998; Christoforidis et al., 2002). However, these strategies although showing promise in some clinical settings, require surgical intervention and their own associated toxicities

The combination of TNF-α with chemotherapeutic agents, such as cisplatin and adriamycin, that damage DNA has demonstrated synergistic effects in experimental models (Duan et al., 2001; Bonavida et al., 1990). Recently, isolated limb perfusion with melphalan, a bi-functional alkylating agent, and TNF-α has been reported to be a successful therapeutic strategy for limb sarcomas and melanomas (Thom et al., 1995). However, systemic toxicities have limited the use of TNF-α in human cancer therapy (Spriggs et al., 1988).

The present invention exemplifies chemoinduction of TNF-α under the control of the inducible Egr-1 promoter, which can be induced by ROI's, damaged DNA and IR, by a chemotherapeutic agent. Studies in mice models of cancer and human cancer cells show that the chemoinduction of TNF-α in itself did not cause any toxicity.

2. Tumor Suppressors

p53. p53 currently is recognized as a tumor suppressor gene. High levels of mutant p53 have been found in many cells transformed by chemical carcinogenesis, ultraviolet radiation, and several viruses. The p53 gene is a frequent target of mutational inactivation in a wide variety of human tumors and is already documented to be the most frequently mutated gene in common human cancers. It is mutated in over 50% of human NSCLC (Hollstein et al., 1991) and in a wide spectrum of other tumors.

The p53 gene encodes a 393-amino acid phosphoprotein that can form complexes with host proteins such as SV40 large-T antigen and adenoviral E1B. The protein is found in normal tissues and cells, but at concentrations which are minute by comparison with transformed cells or tumor tissue. Interestingly, wild-type p53 appears to be important in regulating cell growth and division. Overexpression of wild-type p53 has been shown in some cases to be anti-proliferative in human tumor cell lines. Thus, p53 can act as a negative regulator of cell growth (Weinberg, 1991) and may directly suppress uncontrolled cell growth or indirectly activate genes that suppress this growth. Thus, absence or inactivation of wild-type p53 may contribute to transformation. However, some studies indicate that the presence of mutant p53 may be necessary for full expression of the transforming potential of the gene.

Wild-type p53 is recognized as an important growth regulator in many cell types. Missense mutations are common for the p53 gene and are essential for the transforming ability of the oncogene. A single genetic change prompted by point mutations can create carcinogenic p53, in as much as mutations in p53 are known to abrogate the tumor suppressor capability of wild-type p53. Unlike other oncogenes, however, p53 point mutations are known to occur in at least 30 distinct codons, often creating dominant alleles that produce shifts in cell phenotype without a reduction to homozygosity. Additionally, many of these dominant negative alleles appear to be tolerated in the organism and passed on in the germ line. Various mutant alleles appear to range from minimally dysfunctional to strongly penetrant, dominant negative alleles (Weinberg, 1991).

Casey and colleagues reported that transfection of DNA encoding wild-type p53 into two human breast cancer cell lines restores growth suppression control in such cells (Casey et al., 1991). A similar effect also has been demonstrated on transfection of wild-type, but not mutant, p53 into human lung cancer cell lines (Takahasi et al., 1992). p53 appears dominant over the mutant gene and will select against proliferation when transfected into cells with the mutant gene. Normal expression of the transfected p53 does not affect the growth of normal or non-malignant cells with endogenous p53. Thus, such constructs might be taken up by normal cells without adverse effects. It is thus proposed that the treatment of p53-associated cancers with wild-type p53 will reduce the number of malignant cells or their growth rate. Currently, adenoviral-p53 clinical trials are well under way with excellent results being reported.

p16. The major transitions of the eukaryotic cell cycle are triggered by cyclin-dependent kinases, or CDK's. One CDK, cyclin-dependent kinase 4 (CDK4), regulates progression through the G. The activity of this enzyme may be to phosphorylate Rb at late GI. The activity of CDK4 is controlled by an activating subunit, D-type cyclin, and by an inhibitory subunit p16^{INK4}. The p16^{INK4} has been biochemically characterized as a protein that specifically binds to and inhibits CDK4, and thus may regulate Rb phosphorylation (Serrano et al., 1993; Serrano et al., 1995). Since the p16^{INK4} protein is a CDK4 inhibitor (Serrano, 1993), deletion of this gene may increase the activity of CDK4, resulting in hyperphosphorylation of the Rb protein. p16 also is known to regulate the function of CDK6.

p16^{INK4} belongs to a newly described class of CDK-inhibitory proteins that also includes p15^{INK4B}, p21^{WAF1}, and p27^{KIP1}. The p16^{INK4} gene maps to 9p21, a chromosome region frequently deleted in many tumor types. Homozygous deletions and mutations of the p₁₆^{INK4} gene are frequent in human tumor cell lines. This evidence suggests that the p16^{INK4} gene is a tumor suppressor gene. This interpretation has been challenged, however, by the observation that the frequency of the p16^{INK4} gene alterations is much lower in primary uncultured tumors than in cultured cell lines (Caldas et al., 1994; Cheng et al., 1994; Hussussian et al., 1994; Kamb et al., 1994; Mori et al., 1994; Okamoto et al., 1994; Nobori et al., 1995; Orlow et al., 1994; Arap et al., 1995). However, it was later shown that while the p16 gene was intact in many primary tumors, there were other mechanisms that prevented p16 protein expression in a large percentage of some tumor types. p16 promoter hypermethylation is one of these mechanisms (Merlo et al., 1995; Herman, 1995; Gonzalez-Zulueta, 1995). Restoration of wild-type p16^{INK4} function by transfection with a plasmid expression vector reduced colony formation by some human cancer cell lines (Okamoto, 1994; Arap, 1995). Delivery of p16 with adenovirus vectors inhibits proliferation of some human cancer lines and reduces the growth of human tumor xenografts.

C-CAM. C-CAM is expressed in virtually all epithelial cells (Odin and Obrink, 1987). C-CAM, with an apparent molecular weight of 105 kD, was originally isolated from the plasma membrane of the rat hepatocyte by its reaction with specific antibodies that neutralize cell aggregation (Obrink, 1991). Recent studies indicate that, structurally, C-CAM belongs to the immunoglobulin (Ig) superfamily and its sequence is highly homologous to carcinoembryonic antigen (CEA) (Lin and Guidotti, 1989). Using a baculovirus expression system, Cheung et al. (1993) demonstrated that the first Ig domain of C-CAM is critical for cell adhesive activity.

Cell adhesion molecules, or CAM's are known to be involved in a complex network of molecular interactions that regulate organ development and cell differentiation (Edelman, 1985). Recent data indicate that aberrant expression of CAM's maybe involved in the tumorigenesis of several neoplasms; for example, decreased expression of E-cadherin, which is predominantly expressed in epithelial cells, is associated with the progression of several kinds of neoplasms (Edelman and Crossin, 1991; Frixen et al., 1991; Bussemakers et al., 1992; Matsura et al., 1992; Umbas et al., 1992). Also, Giancotti and Ruoslahti (1990) demonstrated that increasing expression of α₅β₁ integrin by gene transfer can reduce tumorigenicity of Chinese hamster ovary cells in vivo. C-CAM now has been shown to suppress tumor growth in vitro and in vivo.

Other Tumor Suppressors. Other tumor suppressors that may be employed according to the present invention include p21, p15, BRCA1, BRCA2, IRF-1, PTEN, RB, APC, DCC, NF-1, NF-2, WT-1, MEN-I, MEN-II, zac1, p73, VHL, FCC, MCC, DBCCR1, DCP4 and p57.

3. Inducers of Apoptosis

Inducers of apoptosis, such as Bax, Bak, Bcl-Xₛ, Bad, Bim, Bik, Bid, Harakiri, Ad E1B, Bad, ICE-CED3 proteases, TRAIL, SARP-2 and apoptin, similarly could find use according to the present invention.

4. Enzymes

Various enzyme genes are of interest according to the present invention. Such enzymes include cytosine deaminase, adenosine deaminase, hypoxantlhine-guanine phosphoribosyltransferase, and human thymidine kinase.

5. Cytokines, Hormones and Growth Factors

Another class of genes that is contemplated to be inserted into the vectors of the present invention include interleukins and cytokines. These may further include Interleukin 1, IL-2, IL-3, IL-4, IL-5, IL-6, IL-7, IL-8, IL-9, IL-10, IL-11, IL-12, IL-13, IL-14, IL-15, β-interferon, α-interferon, γ-interferon, angiostatin, thrombospondin, endostatin, METH-1, METH-2, GM-CSF, G-CSF, and M-CSF.

6. Toxins

Various toxins are also contemplated to be useful as part of the expression vectors of the present invention, these toxins include bacterial toxins such as ricin A-chain (Burbage, 1997), diphtheria toxin A (Massuda et al., 1997; Lidor, 1997), pertussis toxin A subunit, E. coli enterotoxin toxin A subunit, cholera toxin A subunit and pseudomonas toxin c-terminal. Recently, it was demonstrated that transfection of a plasmid containing the fusion protein regulatable diphtheria toxin A chain gene was cytotoxic for cancer cells. Thus, gene transfer of regulated toxin genes might also be applied to the treatment of cancers (Massuda et al., 1997).

7. Antisense Constructs

Antisense methodology takes advantage of the fact that nucleic acids tend to pair with “complementary” sequences. By complementary, it is meant that polynucleotides are those that are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNA's, may be employed to inhibit gene transcription or translation or both within a host cell, either in vitro or in vivo, such as within a host animal, including a human subject.

Antisense constructs may be designed to bind to the promoter and other control regions, exons, introns or even exon-intron boundaries of a gene. It is contemplated that the most effective antisense constructs will include regions complementary to intron/exon splice junctions. Thus, it is proposed that a preferred embodiment includes an antisense construct with complementarity to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozyme; see below) could be designed. These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

Particular oncogenes that are targets for antisense constructs are ras, myc, neu, raf erb, src, fms, jun, trk, ret, hst, gsp, bcl-2 and abl. Also contemplated to be useful will be anti-apoptotic genes and angiogenesis promoters.

8. Ribozymes

Although proteins traditionally have been used for catalysis of nucleic acids, another class of macromolecules has emerged as useful in this endeavor. Ribozymes are RNA-protein complexes that cleave nucleic acids in a site-specific fashion. Ribozymes have specific catalytic domains that possess endonuclease activity (Kim and Cook, 1987; Gerlach et al., 1987; Forster and Symons, 1987). For example, a large number of ribozymes accelerate phosphoester transfer reactions with a high degree of specificity, often cleaving only one of several phosphoesters in an oligonucleotide substrate (Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, 1989). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al., 1991; Sarver et al., 1990). Recently, it was reported that ribozymes elicited genetic changes in some cells lines to which they were applied; the altered genes included the oncogenes H-ras, c-fos and genes of HIV. Most of this work involved the modification of a target mRNA, based on a specific mutant codon that is cleaved by a specific ribozyme. Targets for this embodiment will include angiogenic genes such as VEGFs and angiopoeiteins as well as the oncogenes (e.g., ras, myc, neu, raf erb, src, fms, jun, trk, ret, hst, gsp, bcl-2, EGFR, grb2 and abl).

9. Single Chain Antibodies

In yet another embodiment, one gene may comprise a single-chain antibody. Methods for the production of single-chain antibodies are well known to those of skill in the art. The skilled artisan is referred to U.S. Pat. No. 5,359,046, (incorporated herein by reference) for such methods. A single chain antibody is created by fusing together the variable domains of the heavy and light chains using a short peptide linker, thereby reconstituting an antigen-binding site on a single molecule.

Single-chain antibody variable fragments (scFvs) in which the C-terminus of one variable domain is tethered to the N-terminus of the other via a 15 to 25 amino acid peptide or linker, have been developed without significantly disrupting antigen binding or specificity of the binding (Bedzyk et al., 1990; Chaudhary et al., 1990). These Fvs lack the constant regions (Fc) present in the heavy and light chains of the native antibody.

Antibodies to a wide variety of molecules are contemplated, such as oncogenes, growth factors, hormones, enzymes, transcription factors or receptors. Also contemplated are secreted antibodies, targeted to serum, against angiogenic factors (VEGF/VSP; βFGF; αFGF) and endothelial antigens necessary for angiogenesis (i.e., V3 integrin). Specifically contemplated are growth factors such as transforming growth factor and platelet derived growth factor.

10. RNA Interference

RNA interference (also referred to as “RNA-mediated interference” or RNAi) is a mechanism by which gene expression can be reduced or eliminated. Double stranded RNA (dsRNA) has been observed to mediate the reduction, which is a multi-step process. dsRNA activates post-transcriptional gene expression surveillance mechanisms that appear to function to defend cells from virus infection and transposon activity. (Fire et al., 1998; Grishok et al., 2000; Ketting et al., 1999; Lin et al., 1999; Montgomery et al., 1998; Sharp et al., 2000; Tabara et al., 1999). Activation of these mechanisms targets mature, dsRNA-complementary mRNA for destruction. RNAi offers major experimental advantages for study of gene function. These advantages include a very high specificity, ease of movement across cell membranes, and prolonged down-regulation of the targeted gene. (Fire et al., 1998; Grishok et al., 2000; Ketting et al., 1999; Lin et al., 1999; Montgomery et al., 1998; Sharp, 1999; Sharp et al., 2000; Tabara et al., 1999). Moreover, dsRNA has been shown to silence genes in a wide range of systems, including plants, protozoans, fungi, C. elegans, Trypanasoma, Drosophila, and mammals (Grishok et al., 2000; Sharp, 1999; Sharp et al., 2000; Elbashir et al., 2001). It is generally accepted that RNAi acts post-transcriptionally, targeting RNA transcripts for degradation. It appears that both nuclear and cytoplasmic RNA can be targeted. (Bosher et al., 2000).

siRNAs must be designed so that they are specific and effective in suppressing the expression of the genes of interest. Methods of selecting the target sequences, i.e. those sequences present in the gene or genes of interest to which the siRNAs will guide the degradative machinery, are directed to avoiding sequences that may interfere with the siRNA's guide function while including sequences that are specific to the gene or genes. Typically, siRNA target sequences of about 21 to 23 nucleotides in length are most effective. This length reflects the lengths of digestion products resulting from the processing of much longer RNAs as described above. (Montgomery et al., 1998).

The making of siRNAs has been mainly through direct chemical synthesis; through processing of longer, double stranded RNAs through exposure to Drosophila embryo lysates; or through an in vitro system derived from S2 cells. Use of cell lysates or in vitro processing may further involve the subsequent isolation of the short, 21-23 nucleotide siRNAs from the lysate, etc., making the process somewhat cumbersome and expensive. Chemical synthesis proceeds by making two single stranded RNA-oligomers followed by the annealing of the two single stranded oligomers into a double stranded RNA. Methods of chemical synthesis are diverse. Non-limiting examples are provided in U.S. Pat. Nos. 5,889,136; 4,415,732; 4,458,066, expressly incorporated herein by reference, and in Wincott et. al. (1995).

Several further modifications to siRNA sequences have been suggested in order to alter their stability or improve their effectiveness. It is suggested that synthetic complementary 21-mer RNAs having di-nucleotide overhangs (i.e., 19 complementary nucleotides + 3′ non-complementary dimers) may provide the greatest level of suppression. These protocols primarily use a sequence of two (2′-deoxy) thymidine nucleotides as the di-nucleotide overhangs. These dinucleotide overhangs are often written as dTdT to distinguish them from the typical nucleotides incorporated into RNA. The literature has indicated that the use of dT overhangs is primarily motivated by the need to reduce the cost of the chemically synthesized RNAs. It is also suggested that the dTdT overhangs might be more stable than UU overhangs, though the data available shows only a slight (<20%) improvement of the dTdT overhang compared to an siRNA with a UU overhang.

Chemically synthesized siRNAs are found to work optimally when they are in cell culture at concentrations of 25-100 nM. This had been demonstrated by Elbashir et. al. wherein concentrations of about 100 nM achieved effective suppression of expression in mammalian cells. siRNAs have been most effective in mammalian cell culture at about 100 nM. In several instances, however, lower concentrations of chemically synthesized siRNA have been used (Caplen et. al., 2000; Elbashir et. al., 2001).

WO 99/32619 and WO 01/68836 suggest that RNA for use in siRNA may be chemically or enzymatically synthesized. Both of these texts are incorporated herein in their entirety by reference. The enzymatic synthesis contemplated in these references is by a cellular RNA polymerase or a bacteriophage RNA polymerase (e.g., T3, T7, SP6) via the use and production of an expression construct as is known in the art. For example, see U.S. Pat. No. 5,795,715. The contemplated constructs provide templates that produce RNAs that contain nucleotide sequences identical to a portion of the target gene. The length of identical sequences provided by these references is at least 25 bases, and may be as many as 400 or more bases in length. An important aspect of this reference is that the authors contemplate digesting longer dsRNAs to 21-25mer lengths with the endogenous nuclease complex that converts long dsRNAs to siRNAs in vivo. They do not describe or present data for synthesizing and using in vitro transcribed 21 -25mer dsRNAs. No distinction is made between the expected properties of chemical or enzymatically synthesized dsRNA in its use in RNA interference.

Similarly, WO 00/44914, incorporated herein by reference, suggests that single strands of RNA can be produced enzymatically or by partial/total organic synthesis. Preferably, single stranded RNA is enzymatically synthesized from the PCR™ products of a DNA template, preferably a cloned cDNA template and the RNA product is a complete transcript of the cDNA, which may comprise hundreds of nucleotides. WO 01/36646, incorporated herein by reference, places no limitation upon the manner in which the siRNA is synthesized, providing that the RNA may be synthesized in vitro or in vivo, using manual and/or automated procedures. This reference also provides that in vitro synthesis may be chemical or enzymatic, for example using cloned RNA polymerase (e.g., T3, T7, SP6) for transcription of the endogenous DNA (or cDNA) template, or a mixture of both. Again, no distinction in the desirable properties for use in RNA interference is made between chemically or enzymatically synthesized siRNA.

U.S. Pat. No. 5,795,715 reports the simultaneous transcription of two complementary DNA sequence strands in a single reaction mixture, wherein the two transcripts are immediately hybridized. The templates used are preferably of between 40 and 100 base pairs, and which is equipped at each end with a promoter sequence. The templates are preferably attached to a solid surface. After transcription with RNA polymerase, the resulting dsRNA fragments may be used for detecting and/or assaying nucleic acid target sequences.

11. Cell Cycle Regulators

Cell cycle regulators provide possible advantages, when combined with other genes. Such cell cycle regulators include p27, p21, p57, p18, p73, p19, p15, E2F-1, E2F-2, E2F-3, p107, p130 and E2F-4. Other cell cycle regulators include anti-angiogenic proteins, such as soluble Flt1 (dominant negative soluble VEGF receptor), soluble Wnt receptors, soluble Tie2/Tek receptor, soluble hemopexin domain of matrix metalloprotease 2 and soluble receptors of other angiogenic cytokines (e.g., VEGFR1/KDR, VEGFR3/Flt4, both VEGF receptors).

12. Chemokines

Genes that code for chemokines also may be used in the present invention. Chemokines generally act as chemoattractants to recruit immune effector cells to the site of chemokine expression. It may be advantageous to express a particular chemokine gene in combination with, for example, a cytokine gene, to enhance the recruitment of other immune system components to the site of treatment. Such chemokines include RANTES, MCAF, MIP1-α, MIP1-β and IP-10. The skilled artisan will recognize that certain cytokines are also known to have chemoattractant effects and could also be classified under the term chemokines.

D. Chemotherapeutics

1. Doxorubicin

Doxorubicin hydrochloride, 5,1 2-Naphthacenedione, (8s-cis)-10-[(3-amino-2,3,6-trideoxy-a-L-lyxo-hexopyranosyl)oxy]-7,8,9,10-tetrahydro-6,8,11-trihydroxy-8-(hydroxyacetyl)-1-methoxy-hydrochloride (hydroxydaunorubicin hydrochloride, adriamycin) is used in a wide antineoplastic spectrum. It binds to DNA and inhibits nucleic acid synthesis, inhibits mitosis and promotes chromosomal aberrations.

Administered alone, it is the drug of first choice for the treatment of thyroid adenoma and primary hepatocellular carcinoma. It is a component of 31 first-choice combinations for the treatment of ovarian, endometrial and breast tumors, bronchogenic oat-cell carcinoma, non-small cell lung carcinoma, gastric adenocarcinoma, retinoblastoma, neuroblastoma, mycosis fungoides, pancreatic carcinoma, prostatic carcinoma, bladder carcinoma, myeloma, diffuse histiocytic lymphoma, Wilms' tumor, Hodgkin's disease, adrenal tumors, osteogenic sarcoma soft tissue sarcoma, Ewing's sarcoma, rhabdomyosarcoma and acute lymphocytic leukemia. It is an alternative drug for the treatment of islet cell, cervical, testicular and adrenocortical cancers. It is also an immunosuppressant.

Doxorubicin is absorbed poorly and must be administered intravenously. The pharmacokinetics are multicompartmental. Distribution phases have half-lives of 12 minutes and 3.3 hr. The elimination half-life is about 30 hr. Forty to 50% is secreted into the bile. Most of the remainder is metabolized in the liver, partly to an active metabolite (doxorubicinol), but a few percent is excreted into the urine. In the presence of liver impairment, the dose should be reduced.

Appropriate doses are, intravenous, adult, 60 to 75 mg/m² at 21-day intervals or 25 to 30 mg/m² on each of 2 or 3 successive days repeated at 3- or 4-wk intervals or 20 mg/m² once a week. The lowest dose should be used in elderly patients, when there is prior bone-marrow depression caused by prior chemotherapy or neoplastic marrow invasion, or when the drug is combined with other myelopoietic suppressant drugs. The dose should be reduced by 50% if the serum bilirubin lies between 1.2 and 3. mg/dL and by 75% if above 3 mg/dL. The lifetime total dose should not exceed 550 mg/m² in patients with normal heart function and 400 mg/m² in persons having received mediastinal irradiation. Alternatively, 30 mg/m² on each of 3 consecutive days, repeated every 4 wk. Exemplary doses may be 10 mg/m², 20 mg/m², 30 mg/m², 50 mg/m², 100 mg/m², 150 mg/m², 175 mg/m², 200 mg/m², 225 mg/m², 250 mg/m², 275 mg/m², 300 mg/m², 350 mg/m², 400 mg/m², 425 mg/m², 450 mg/m², 475 mg/m², 500 mg/m². Of course, all of these dosages are exemplary, and any dosage in-between these points is also expected to be of use in the invention.

2. Cyclophosphamide

Cyclophosphamide is 2H-1,3,2-Oxazaphosphorin-2-amine, N,N-bis(2-chloroethyl)tetrahydro-, 2-oxide, monohydrate; termed Cytoxan available from Mead Johnson; and Neosar available from Adria. Cyclophosphamide is prepared by condensing 3-amino-1-propanol with N,N-bis(2-chlorethyl) phosphoramidic dichloride [(ClCH₂ CH₂)₂N—POCl₂] in dioxane solution under the catalytic influence of triethylamine. The condensation is double, involving both the hydroxyl and the amino groups, thus effecting the cyclization. Unlike other β-chloroethylamino alkylators, it does not cyclize readily to the active ethyleneimonium form until activated by hepatic enzymes. Thus, the substance is stable in the gastrointestinal tract, tolerated well and effective by the oral and parental routes and does not cause local vesication, necrosis, phlebitis or even pain.

Suitable doses for adults include, orally, 1 to 5 mg/kg/day (usually in combination), depending upon gastrointestinal tolerance; or 1 to 2 mg/kg/day; intravenously, initially 40 to 50 mg/kg in divided doses over a period of 2 to 5 days or 10 to 15 mg/kg every 7 to 10 days or 3 to 5 mg/kg twice a week or 1.5 to 3 mg/kg/day. A dose 250 mg/kg/day may be administered as an antineoplastic. Because of gastrointestinal adverse effects, the intravenous route is preferred for loading. During maintenance, a leukocyte count of 3000 to 4000/mm³ usually is desired. The drug also sometimes is administered intramuscularly, by infiltration or into body cavities. It is available in dosage forms for injection of 100, 200 and 500 mg, and tablets of 25 and 50 mg the skilled artisan is referred to “Remington's Pharmaceutical Sciences” 15th Edition, chapter 61, incorporate herein as a reference, for details on doses for administration.

3. 5-Fluorouracil

5-Fluorouracil (5-FU) has the chemical name of 5-fluoro-2,4(1H,3H)-pyrimidinedione. Its mechanism of action is thought to be by blocking the methylation reaction of deoxyuridylic acid to thymidylic acid. Thus, 5-FU interferes with the syntheisis of deoxyribonucleic acid (DNA) and to a lesser extent inhibits the formation of ribonucleic acid (RNA). Since DNA and RNA are essential for cell division and proliferation, it is thought that the effect of 5-FU is to create a thymidine deficiency leading to cell death. Thus, the effect of 5-FU is found in cells that rapidly divide, a characteristic of metastatic cancers.

E. Expression Constructs

The present invention will involve the generation and use of expression constructs containing, for example, a tumor therapeutic gene and a means for its expression, replicating the vector in an appropriate helper cell, obtaining viral particles produced therefrom, and infecting cells with the recombinant viral particles. The following discussion is directed toward engineering expression constructs for recombinant protein production and/or gene therapy. The gene may be a therapeutic gene that encodes a TNF-α protein, and may further include a second therapeutic gene for use in adjunct therapy.

The present invention contemplates transferring an expression construct comprising a promoter operatively linked to a nucleic acid encoding, e.g., TNF-α into a cell.

1. Promoters and Enhancers

A “promoter” is a control sequence that is a region of a nucleic acid sequence at which initiation and rate of transcription are controlled. It may contain genetic elements at which regulatory proteins and molecules may bind such as RNA polymerase and other transcription factors. As used herein, the term “promoter” includes what is referred to in the art as an upstream promoter region, a promoter region or a promoter of a generalized eukaryotic RNA Polymerase II transcription unit. Exemplary and preferred promoters are the TATA box, the CAAT box and GC-rich sequence elements.

The phrases “operatively positioned,” “operatively linked,” “under control,” and “under transcriptional control,” as in the present invention, mean that a promoter is in a correct functional location and/or orientation in relation to a nucleic acid encoding, e.g., TNF-α, to control transcriptional initiation and/or expression of, e.g., TNF-α.

The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous. A promoter may or may not be used in conjunction with an enhancer.

An enchancer is another type of discrete transcription regulatory sequence element. An enhancer provides specificity of time, location and expression level for a particular encoding region (e.g., gene). A major function of an enhancer is to increase the level of transcription of an encoding region in a cell that contains one or more transcription factors that bind to that enhancer. Unlike a promoter, an enhancer can function when located at variable distances from transcription start sites so long as a promoter is present.

Naturally, it may be important to employ a promoter and/or enhancer that effectively directs the expression of the nucleic acid segment in the cell type, organelle, and organism chosen for expression. Those of skill in the art of molecular biology generally know the use of promoters, enhancers, and cell type combinations for protein expression, for example, see Sambrook et al. (2000), incorporated herein by reference. The present invention particularly utilizes promoters responsive to chemotherapeutic agents such as doxorubicin, cyclophosphamide and/or 5-FU. Examples of promoters for use in the present invention, include but are not limited to, an Egr-1 promoter, a c-fos or a c-Jun promoter.

a. Egr-1 Promoter

Exposure of mammalian cells to ionizing radiation is associated with induction of Egr-1 gene expression. The Egr-1 gene (also known as zif/268, TIS-8, NFGI-A and Krox-24; Sukhatme, et al. 1988; Christy, et al., 1988; Milbrandt, 1987; Lemaire, et al., 1988; Gessler, 1990) encodes a 533-amino acid residue nuclear phosphoprotein with a Cys₂ -His₂ zinc finger domain that is partially homologous to the corresponding domain in the Wilms' tumor-susceptibility gene (Gessler, 1990). The Egr-1 protein binds to the DNA sequence CGCCCCCGC in a zinc-dependent manner and functions as a regulator of gene transcription (Christy, et al., 1988; Cao, et al., 1990). The Egr-1 promoter region contains several putative cis elements including six CArG domains (Christy, et al., 1988; Qureshi, et al., 1991). Although studies have supported the involvement of CArG domains in x-ray induced Egr-1 transcription, other sequences between these domains may also serve as functional cis elements.

The, x-ray inducibility of the Egr-1 gene was conferred by a region of the Egr-1 promoter that contains CArG domains. The six CArG domains of the Egr-1 promoter are located within a region of the Egr-1 promoter located about 960 nucleotide bases upstream from the transcription initiation site of the Egr-1 gene. A single CArG domain was shown to be sufficient to confer radiation inducibility. Preferably, a radiation responsive enhancer-promoter comprises at least one of the three most distal (i.e. upstream) CArG domains. Both mitogenic and differentiation signals have been shown to induce the rapid and transient expression of Egr-1 in a variety of cell types.

b. c-Fos Promoter

Studies with the c-fos promoter have demonstrated that the CArG domain or serum response element is functional in inducing transcription of this gene in response to serum and other signals (Triesman, 1990). The CArG element is required for c-fos induction by both PKC-mediated signaling pathways and by growth factor-induced signals independent of PKC (Fisch, et al., 1987; Gilman, 1988; Buscher, et al., 1988; Sheng, et al., 1988; Stumpo, et al., 1988). The kinetics of induction, as well as repression, of c-fos expression are similar to those of Egr-1 in other models (Sukhatme, et al., 1988). Indeed, x-ray-induced changes in c-fos transcripts are similar to those obtained for Egr-1 in HL-525 cells and TPA-induced c-fos expression, like that for Egr-1, is attenuated in these cells. Studies with the c-fos promoter have demonstrated that the CArG domain functions as a binding site for the serum response factor (SRF) (Treisman, 1986; Prywes, et al., 1988). SRF binds, but with varying affinity, to the different CArG elements in the Egr-1 promoter (Christy, et al., 1988).

c. c-Jun Promoter

Exposure of cells to x-rays is associated with activation of the c-Jun/c-fos gene families, which encode transcription factors (Hallahan et al., 1991; Sherman et al., 1990). The c-Jun gene encodes the major form of the 40-44 kD AP-1 transcription factor (Mitchell, et al., 1989). The Jun/AP-1 complex binds to the heptomeric DNA consensus sequence TGA^{G}/_{C}TCA (Mitchell, et al., 1989). The DNA binding domain of c-Jun is shared by a family of transcription factors, including Jun-B, Jun-D and c-fos. Moreover, the affinity of c-Jun binding to DNA is related to the formation of homodimers or heterodimers with products of the fos gene family (Nakabeppa, et al., 1988; Halazonetis, et al., 1988).

Phorbol ester activation of c-Jun transcription in diverse cell types has implicated the involvement of a protein kinase C (PKC)-dependent mechanism (Brenner, et al., 1989; Angel, et al., 1988; Hallahan, et al., 1991). A similar pathway likely plays a role, at least in part, in the induction of c-Jun expression by ionizing radiation. Prolonged treatment with phorbol esters to down-regulate PKC is associated with decreases in the effects of x-rays on c-Jun transcription (Hallahan, et al., 1991). Furthermore, non-specific inhibitors of PKC, such as the isoquinolinesulfonamide derivative, H7, block x-ray-induced c-Jun gene product expression (Hallahan, et al., 1991).

The effects of ionizing radiation on c-Jun gene product expression were studied in an HL-60 cell variant, designated HL-525, which variant is deficient in PKC-mediated signal transduction (Homma et al., 1986). That variant was resistant to both phorbol ester-induced differentiation and x-ray-induced TNF gene product expression (Hallahan et al., 1991; Homma et al., 1986) and resistant to the induction of c-Jun gene product expression by phorbol esters.

2. Other Elements

a. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome-scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well as an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, herein incorporated by reference).

b. Multiple Cloning and Splicing Sites

Vectors can include a multiple cloning site (MCS), which is a nucleic acid region that contains multiple restriction enzyme sites, any of which can be used in conjunction with standard recombinant technology to digest the vector (see Carbonelli et al., 1999, Levenson et al., 1998, and Cocea, 1997, incorporated herein by reference). “Restriction enzyme digestion” refers to catalytic cleavage of a nucleic acid molecule with an enzyme which functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (see Chandler et al., 1997, incorporated herein by reference).

c. Termination and Polyadenylation Signals and Origins of Replication

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 “A” residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and/or to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

In expression, particularly eukaryotic expression, one will typically include a polyadenylation signal to effect proper polyadenylation of the transcript. Polyadenylation may increase the stability of the transcript or may facilitate cytoplasmic transport.

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

d. Selectable and Screenable Markers

In certain embodiments of the invention, cells containing an expression construct may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually, the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

F. Delivery of Nucleic Acids

Nucleic acids of the present invention are delivered to a cell in order to mediate and intended effect, which may or may not include transcription or translation. Such delivery, as contemplated in the present invention, may employ viral or non-viral vectors.

1. Viral Vector-Mediated Delivery

The ability of certain viruses to infect cells or enter cells via receptor-mediated endocytosis, and to integrate into host cell genome and express viral genes stably and efficiently have made them attractive candidates for the transfer of foreign nucleic acids into cells (e.g., mammalian cells). Non-limiting examples of virus vectors that may be used to deliver a nucleic acid of the present invention are described below.

a. Adenoviral Vectors

A particular method for delivery of the nucleic acid involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a tissue or cell-specific construct that has been cloned therein. Knowledge of the genetic organization or adenovirus, a 36 kb, linear, double-stranded DNA virus, allows substitution of large pieces of adenoviral DNA with foreign sequences up to 7 kb (Grunhaus and Horwitz, 1992).

b. AAV Vectors

The nucleic acid may be introduced into the cell using adenovirus assisted transfection. Increased transfection efficiencies have been reported in cell systems using adenovirus coupled systems (Kelleher and Vos, 1994; Cotten et al., 1992; Curiel, 1994). Adeno-associated virus (AAV) is an attractive vector system for use according to the present invention as it has a high frequency of integration and it can infect nondividing cells, thus making it useful for delivery of genes into mammalian cells, for example, in tissue culture (Muzyczka, 1992) or in vivo. AAV has a broad host range for infectivity (Tratschin et al., 1984; Laughlin et al., 1986; Lebkowski et al., 1988; McLaughlin et al., 1988). Details concerning the generation and use of rAAV vectors are described in U.S. Pat. Nos. 5,139,941 and 4,797,368, each incorporated herein by reference.

c. Retroviral Vectors

Retroviruses have promise as gene delivery vectors due to their ability to integrate their genes into the host genome, transferring a large amount of foreign genetic material, infecting a broad spectrum of species and cell types and of being packaged in special cell-lines (Miller, 1992).

In order to construct a retroviral vector, a nucleic acid is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into a special cell line (e.g., by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require.the division of host cells (Paskind et al., 1975).

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; U.S. Pat. Nos. 6,013,516 and 5,994,136). Some examples of lentivirus include the Human Immunodeficiency Viruses (HIV-1, HIV-2) and the Simian Immunodeficiency Virus (SIV). Lentiviral vectors have been generated by multiply attenuating the HIV virulence genes, for example, the genes env, vif, vpr, vpu and nef are deleted making the vector biologically safe.

Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. One may target the recombinant virus by linkage of the envelope protein with an antibody or a particular ligand for targeting to a receptor of a particular cell-type. By inserting a sequence (including a regulatory region) of interest into the viral vector, along with another gene that encodes the ligand for a receptor on a specific target cell, the vector is now target-specific.

d. Other Viral Vectors

Other viral vectors may be employed as vaccine constructs in the present invention. Vectors derived from viruses such as vaccinia virus (Ridgeway, 1988; Baichwal and Sugden, 1986; Coupar et al., 1988), sindbis virus, cytomegalovirus and herpes simplex virus may be employed. They offer several attractive features for various mammalian cells (Friedmann, 1989; Ridgeway, 1988; Baichwal and Sugden, 1986; Coupar et al., 1988; Horwich et al., 1990).

e. Delivery Using Modified Viruses

A nucleic acid to be delivered may be housed within an infective virus that has been engineered to express a specific binding ligand. The virus particle will thus bind specifically to the cognate receptors of the target cell and deliver the contents to the cell. A novel approach designed to allow specific targeting of retrovirus vectors was developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

2. Non-viral Delivery

Several non-viral methods for the transfer of expression constructs are contemplated by the present invention. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979), cell sonication (Fechheimer et al., 1987), gene bombardment using high velocity microprojectiles (Yang et al., 1990), and receptor-mediated transfection (Wu and Wu, 1987; Wu and Wu, 1988).

Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. There are numerous U.S. Patent references describing pharmaceutical delivery employing liposomes. For example, U.S. Pat. No. 5,762,904, incorporated herein by reference, describes the use of polymerized liposomes, methods of preparing the polymerized liposomes and incorporating biologically active substances within the polymerized liposomes, and methods of administering polymerized liposomes containing a biologically active substance to be delivered to a patient are discussed. Additional polymerized vesicles are further described in U.S. Pat. No. 4,587,055 specifically incorporated herein by reference. Viral liposome particles are described in detail in U.S. Pat. No. 4,201,767, specifically incorporated herein by reference. U.S. Pat. No. 5,759,566 is incorporated herein by reference and describes liposomic dispersions containing proteinaceous substances, which allow the systemic, local or topical administration of drugs by transmucosal route are described. There are numerous other U.S. Patents that describe the use of liposomes for a therapeutic delivery, as such the use of liposomal delivery of the nucleic acid and or protein compositions of the present invention are well within the skill of the art.

Other vector delivery systems, which can be employed to deliver a nucleic acid encoding a therapeutic gene into cells, are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993).

The expression construct may simply consist of naked recombinant DNA or plasmids (Dubensky et al. 1984, Benvenisty and Neshif 1986)). Transfer of the construct may be performed by any of the methods mentioned above which physically or chemically permeabilize the cell membrane.

Another embodiment of the invention for transferring a naked DNA expression construct into cells may involve particle bombardment. This method depends on the ability to accelerate DNA coated microprojectiles to a high velocity allowing them to pierce cell membranes and enter cells without killing them (Klein et al., 1987).

G. Administration and Treatment Regimens of TNF and a Chemotherapeutic Agent

1. Administration

To kill cells, inhibit cell growth, inhibit metastasis, decrease tumor or tissue size and otherwise reverse or reduce the malignant phenotype of tumor cells, using the methods and compositions of the present invention, one will contact a hyperproliferative cell with the therapeutic composition comprising an expression construct encoding a tumor therapeutic gene and doxorubicin, cyclophosphamide, 5-FU, taxol or gemcitabine. The routes of administration will vary, naturally, with the location and nature of the lesion, and include, e.g., intradermal, transdermal, parenteral, intravenous, intramuscular, intranasal, subcutaneous, percutaneous, intratracheal, intraperitoneal, intratumoral, perfusion, lavage, direct injection, and oral administration and formulation.

To effect a therapeutic benefit with respect to a hyperproliferataive condition or disease, one would contact a hyperproliferative cell with the therapeutic compound. Any of the formulations and routes of administration discussed with respect to the treatment or diagnosis of cancer may also be employed with respect to hyperproliferative diseases and conditions.

Intratumoral injection or injection into the tumor vasculature is specifically contemplated for discrete, solid, accessible tumors. Local, regional or systemic administration also may be appropriate. For tumors of >4 cm, the volume to be administered will be about 4-10 ml (preferably 10 ml), while for tumors of <4 cm, a volume of about 1-3 ml will be used (preferably 3 ml). Multiple injections delivered as single dose comprise about 0.1 to about 0.5 ml volumes. The viral particles may advantageously be contacted by administering multiple injections to the tumor, spaced at approximately 1 cm intervals.

In the case of surgical intervention, the present invention may be used preoperatively, to render an inoperable tumor subject to resection. Alternatively, the present invention may be used at the time of surgery, and/or thereafter, to treat residual, recurrent or metastatic disease. For example, a resected tumor bed may be injected or perfused with a formulation comprising an expression construct encoding TNF-α and a chemotherapeutic agent. The perfusion may be continued post-resection, for example, by leaving a catheter implanted at the site of the surgery. Periodic post-surgical treatment also is envisioned.

Continuous administration also may be applied where appropriate, for example, where a tumor is excised and the tumor bed is treated to eliminate residual, microscopic disease. Delivery via syringe or catherization is preferred. Such continuous perfusion may take place for a period from about 1-2 hours, to about 2-6 hours, to about 6-12 hours, to about 12-24 hours, to about 1-2 days, to about 1-2 wk or longer following the initiation of treatment. Generally, the dose of the therapeutic composition via continuous perfusion will be equivalent to that given by a single or multiple injections, adjusted over a period of time during which the perfusion occurs. It is further contemplated that limb perfusion may be used to administer therapeutic compositions of the present invention, particularly in the treatment of melanomas and sarcomas.

2. Treatment Regimens

Treatment regimens may vary as well, and often depend on tumor type, tumor location, disease progression, and health and age of the patient. Obviously, certain types of tumor will require more aggressive treatment, while at the same time, certain patients cannot tolerate more taxing protocols. The clinician will be best suited to make such decisions based on the known efficacy and toxicity (if any) of the therapeutic formulations.

In certain embodiments, the tumor being treated may not, at least initially, be resectable. Treatments with therapeutic viral constructs, such as in the present invention, may increase the resectability of the tumor due to shrinkage at the margins or by elimination of certain particularly invasive portions. Following treatments, resection may be possible. Additional treatments subsequent to resection will serve to eliminate microscopic residual disease at the tumor site.

A typical course of treatment, for a primary tumor or a post-excision tumor bed, will involve multiple doses. Typical primary tumor treatment involves a 6 dose application over a two-week period. The two-week regimen may be repeated one two, three, four, five, six or more times. During a course of treatment, the need to complete the planned dosings may be re-evaluated.

The treatments may include various “unit doses.” Unit dose is defined as containing a predetermined-quantity of the therapeutic composition. The quantity to be administered, and the particular route and formulation, are within the skill of those in the clinical arts. A unit dose need not be administered as a single injection, but may comprise continuous infusion over a set period of time. Unit dose of the present invention may conveniently be described in terms of plaque forming units (pfu) for a viral construct. Unit doses range from 10³, 10⁴, 10⁵, 10⁶, 10⁷, 10⁸, 10⁹, 10¹⁰, 10¹¹, 10¹², 10¹³ pfu and higher. Alternatively, depending on the kind of virus and the titer attainable, one will deliver 1 to 100, 10 to 50, 100-1000, or up to about 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, or 1×10¹⁵ or higher infectious viral particles (vp) to the patient or to the patient's cells.

H. Adjunct Therapies

As discussed above, the present invention may be used in the context of hyperproliferative diseases/conditions including cancer. In order to increase the effectiveness of a treatment with the compositions of the present invention, such as a expression construct comprising a nucleic acid encoding a tumor therapeutic gene and doxorubicin, cyclophosphamide, 5-FU, taxol or gemcitabine, it may be desirable to further combine these compositions with adjunct agents effective in the treatment of those diseases and conditions.

Various combinations may be employed; for example, a nucleic acid encoding a tumor therapeutic gene and chemotherapeutic (doxorubicin, cyclophosphamide, 5-FU, taxol or gemcitabine) is “A,” and the adjunct agent/therapy is “B”:

A/B/A B/A/B B/B/A A/A/B A/B/B B/A/A A/B/B/B
B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of the therapeutic composition comprising a tumor therapeutic gene and chemotherapeutic of the present invention to a patient will follow general protocols for the administration of that particular secondary therapy, taking into account the toxicity, if any, of the treatment. It is expected that the treatment cycles would be repeated as necessary. It also is contemplated that various cancer therapies, such as a second chemotherapy, radiotherapy, as well as surgical intervention, may be applied in combination with the described hyperproliferative or cancer cell therapy, as discussed below.

1. Second Chemotherapy

In particular embodiments of the present invention, a therapeutic expression construct encoding TNF-α may be administered as an anti-hyperproliferative therapy (therapy targeting hyperproliferation in order to suppress, reduce, inhibit, amerliorate, or prevent hyperproliferation of a cell) in combination with a chemotherapeutic (e.g., doxorubicin, cyclophosphamide, 5-FU, taxol or gemcitabine). In addition, the present invention may further include adjunct therapies such as a second chemotherapy.

The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, corticosteroid hormones, mitotic inhibitors, and nitrosoureas, and any analog or derivative variant thereof. It is contemplated that the nucleic acid encoding TNF-α of the present invention may be use in combination with any of the chemotherapeutic agents as a therapeutic composition in treating cancers.

Secondary chemotherapeutics contemplated in the present invention include, for example, cisplatin (CDDP), carboplatin, procarbazine, mechlorethamine, camptothecin, ifosfamide, melphalan, chlorambucil, bisulfan, nitrosurea, dactinomycin, daunorubicin, bleomycin, plicomycin, mitomycin, etoposide (VP16), tamoxifen, transplatinum, vincristine, vinilastine and methotrexate or any analog or derivative variant thereof, but is not limited to such.

a. Alkylating agents

Alkylating agents are drugs that directly interact with genomic DNA to prevent the cancer cell from proliferating. This category of chemotherapeutic drugs represents agents that affect all phases of the cell cycle, that is, they are not phase-specific. Alkylating agents can be implemented to treat chronic leukemia, non-Hodgkin's lymphoma, Hodgkin's disease, multiple myeloma, and particular cancers of the breast, lung, and ovary. They include: busulfan, chlorambucil, cisplatin, dacarbazine, ifosfamide, mechlorethamine (MUSTARGEN), and melphalan. Troglitazalone can be used to treat cancer in combination with any one or more of these alkylating agents, some of which are discussed herein.

i. Busulfan

Busulfan (also known as MYLERAN) is a bifunctional alkylating agent. Busulfan is known chemically as 1,4-butanediol dimethanesulfonate. Busulfan is not a structural analog of the nitrogen mustards. Busulfan is available in tablet form for oral administration. Each scored tablet contains 2 mg busulfan and the inactive ingredients magnesium stearate and sodium chloride. Busulfan is indicated for the palliative treatment of chronic myelogenous (myeloid, myelocytic, granulocytic) leukemia. Although not curative, busulfan reduces the total granulocyte mass, relieves symptoms of the disease, and improves the clinical state of the patient. Approximately 90% of adults with previously untreated chronic myelogenous leukemia will obtain hematologic remission with regression or stabilization of organomegaly following the use of busulfan. It has been shown to be superior to splenic irradiation with respect to survival times and maintenance of hemoglobin levels, and to be equivalent to irradiation at controlling splenomegaly.

ii. Chlorambucil

Chlorambucil (also known as LEUKERAN) is a bifunctional alkylating agent of the nitrogen mustard type that has been found active against selected human neoplastic diseases. Chlorambucil is known chemically as 4-[bis(2-chlorethyl)amino] benzenebutanoic acid. Chlorambucil is available in tablet form for oral administration. It is rapidly and completely absorbed from the gastrointestinal tract. After single oral doses of 0.6-1.2 mg/kg, peak plasma chlorambucil levels are reached within one hour and the terminal half-life of the parent drug is estimated at 1.5 hours. 0.1 to 0.2 mg/kg/day or 3 to 6 mg/m²/day or alternatively 0.4 mg/kg may be used for antineoplastic treatment. Chlorambucil is indicated in the treatment of chronic lymphatic (lymphocytic) leukemia, malignant lymphomas including lymphosarcoma, giant follicular lymphoma and Hodgkin's disease. It is not curative in any of these disorders but may produce clinically useful palliation.

iii. Cisplatin

Cisplatin has been widely used to treat cancers such as metastatic testicular or ovarian carcinoma, advanced bladder cancer, head or neck cancer, cervical cancer, lung cancer or other tumors. Cisplatin can be used alone or in combination with other agents, with efficacious doses used in clinical applications of 15-20 mg/m² for 5 days every three weeks for a total of three courses. Exemplary doses may be 0.50 mg/m², 1.0 mg/ m², 1.50 mg/m², 1.75 mg/m², 2.0 mg/m², 3.0 mg/m², 4.0 mg/m², 5.0 mg/m², 10 mg/m². Of course, all of these dosages are exemplary, and any dosage in-between these points is also expected to be of use in the invention. Cisplatin is not absorbed orally and must therefore be delivered via injection intravenously, subcutaneously, intratumorally or intraperitoneally.

iv. Melphalan

Melphalan, also known as ALKERAN, L-phenylalanine mustard, phenylalanine mustard, L-PAM, or L-sarcolysin, is a phenylalanine derivative of nitrogen mustard. Melphalan is a bifunctional alkylating agent which is active against selective human neoplastic diseases. It is known chemically as 4-[bis(2-chloroethyl)amino]-L-phenylalanine. Melphalan is the active L-isomer of the compound and was first synthesized in 1953 by Bergel and Stock; the D-isomer, known as medphalan, is less active against certain animal tumors, and the dose needed to produce effects on chromosomes is larger than that required with the L-isomer. The racemic (DL-) form is known as merphalan or sarcolysin. Melphalan is insoluble in water and has a pKa₁ of about 2.1. Melphalan is available in tablet form for oral administration and has been used to treat multiple myeloma.

Available evidence suggests that about one third to one half of the patients with multiple myeloma show a favorable response to oral administration of the drug. Melphalan has been used in the treatment of epithelial ovarian carcinoma. One commonly employed regimen for the treatment of ovarian carcinoma has been to administer melphalan at a dose of 0.2 mg/kg daily for five days as a single course. Courses are repeated every four to five weeks depending upon hematologic tolerance (Smith and Rutledge, 1975; Young et al., 1978). Alternatively the dose of melphalan used could be as low as 0.05 mg/kg/day or as high as 3 mg/kg/day or any dose in between these doses or above these doses. Some variation in dosage will necessarily occur depending on the condition of the subject being treated. The person responsible for administration will, in any event, determine the appropriate dose for the individual subject.

b. Antimetabolites

Antimetabolites disrupt DNA and RNA synthesis. Unlike alkylating agents, they specifically influence the cell cycle during S phase. They have used to combat chronic leukemias in addition to tumors of breast, ovary and the gastrointestinal tract. Antimetabolites include cytarabine (Ara-C), fludarabine, gemcitabine, and methotrexate.

c. Antitumor Antibiotics

Antitumor antibiotics have both antimicrobial and cytotoxic activity. These drugs also interfere with DNA by chemically inhibiting enzymes and mitosis or altering cellular membranes. These agents are not phase specific so they work in all phases of the cell cycle. Thus, they are widely used for a variety of cancers. Examples of antitumor antibiotics include bleomycin, dactinomycin, daunorubicin, and idarubicin, some of which are discussed in more detail below. Widely used in clinical setting for the treatment of neoplasms these compounds are administered through bolus injections intravenously at doses ranging from 25-75 mg/m² at 21 day intervals for adriamycin, to 35-100 mg/m² for etoposide intravenously or orally.

i. Daunorubicin

Daunorubicin hydrochloride, 5,12-Naphthacenedione, (8S-cis)-8-acetyl-10-[(3-amino-2,3,6-trideoxy-a-L-lyxo-hexanopyranosyl)oxy]-7,8,9,10-tetrahydro-6,8,11-trihydroxy-10-methoxy-, hydrochloride; also termed CERUBIDINE and available from Wyeth. Daunorubicin intercalates into DNA, blocks DNA directed RNA polymerase and inhibits DNA synthesis. It can prevent cell division in doses that do not interfere with nucleic acid synthesis.

In combination with other drugs it is included in the first-choice chemotherapy of acute myelocytic leukemia in adults (for induction of remission), acute lymphocytic leukemia and the acute phase of chronic myelocytic leukemia. Oral absorption is poor, and it must be given intravenously. The half-life of distribution is 45 minutes and of elimination, about 19 hr. The half-life of its active metabolite, daunorubicinol, is about 27 hr. Daunorubicin is metabolized mostly in the liver and also secreted into the bile (ca 40%). Dosage must be reduced in liver or renal insufficiencies.

Suitable doses are (base equivalent), intravenous adult, younger than 60 yr. 45 mg/m²/day (30 mg/m² for patients older than 60 yr.) for 1, 2 or 3 days every 3 or 4 wk or 0.8 mg/kg/day for 3 to 6 days every 3 or 4 wk; no more than 550 mg/m² should be given in a lifetime, except only 450 mg/m² if there has been chest irradiation; children, 25 mg/m² once a week unless the age is less than 2 yr. or the body surface less than 0.5 m, in which case the weight-based adult schedule is used. It is available in injectable dosage forms (base equivalent) 20 mg (as the base equivalent to 21.4 mg of the hydrochloride). Exemplary doses may be 10 mg/m², 20 mg/m², 30 mg/m², 50 mg/m², 100 mg/m², 150 mg/m², 175 mg/m², 200 mg/m², 225 mg/m², 250 mg/m², 275 mg/m², 300 mg/m², 350 mg/m², 400 mg/m², 425 mg/m², 450 mg/m², 475 mg/m², 500 mg/m². Of course, all of these dosages are exemplary, and any dosage in-between these points is also expected to be of use in the present invention.

ii. Mitomycin

Mitomycin (also known as MUTAMYCIN and/or mitomycin-C) is an antibiotic isolated from the broth of Streptomyces caespitosus which has been shown to have antitumor activity. The compound is heat stable, has a high melting point, and is freely soluble in organic solvents. Mitomycin selectively inhibits the synthesis of deoxyribonucleic acid (DNA). The guanine and cytosine content correlates with the degree of mitomycin-induced cross-linking. At high concentrations of the drug, cellular RNA and protein synthesis are also suppressed.

In humans, mitomycin is rapidly cleared from the serum after intravenous administration. Time required to reduce the serum concentration by 50% after a 30 mg. bolus injection is 17 minutes. After injection of 30 mg, 20 mg, or 10 mg I.V., the maximal serum concentrations were 2.4 mg/mL, 1.7 mg/mL, and 0.52 mg/mL, respectively. Clearance is effected primarily by metabolism in the liver, but metabolism occurs in other tissues as well. The rate of clearance is inversely proportional to the maximal serum concentration because, it is thought, of saturation of the degradative pathways. Approximately 10% of a dose of mitomycin is excreted unchanged in the urine. Since metabolic pathways are saturated at relatively low doses, the percent of a dose excreted in urine increases with increasing dose. In children, excretion of intravenously administered mitomycin is similar.

iii. Actinomycin D

Actinomycin D (Dactinomycin) [50-76-0]; C₆₂ H₈₆ N₁₂ O₁₆ (1255.43) is an antineoplastic drug that inhibits DNA-dependent RNA polymerase. It is a component of first-choice combinations for treatment of choriocarcinoma, embryonal rhabdomyosarcoma, testicular tumor and Wilms' tumor. Tumors that fail to respond to systemic treatment sometimes respond to local perfusion. Dactinomycin potentiates radiotherapy. It is a secondary (efferent) immunosuppressive.

Actinomycin D is used in combination with primary surgery, radiotherapy, and other drugs, particularly vincristine and cyclophosphamide. Antineoplastic activity has also been noted in Ewing's tumor, Kaposi's sarcoma, and soft-tissue sarcomas. Dactinomycin can be effective in women with advanced cases of choriocarcinoma. It also produces consistent responses in combination with chlorambucil and methotrexate in patients with metastatic testicular carcinomas. A response may sometimes be observed in patients with Hodgkin's disease and non-Hodgkin's lymphomas. Dactinomycin has also been used to inhibit immunological responses, particularly the rejection of renal transplants.

Half of the dose is excreted intact into the bile and 10% into the urine; the half-life is about 36 hr. The drug does not pass the blood-brain barrier. Actinomycin D is supplied as a lyophilized powder (0/5 mg in each vial). The usual daily dose is 10 to 15 mg/kg; this is given intravenously for 5 days; if no manifestations of toxicity are encountered, additional courses may be given at intervals of 3 to 4 weeks. Daily injections of 100 to 400 mg have been given to children for 10 to 14 days; in other regimens, 3 to 6 mg/kg, for a total of 125 mg/kg, and weekly maintenance doses of 7.5 mg/kg have been used. Although it is safer to administer the drug into the tubing of an intravenous infusion, direct intravenous injections have been given, with the precaution of discarding the needle used to withdraw the drug from the vial in order to avoid subcutaneous reaction. Exemplary doses may be 100 mg/m², 150 mg/m², 175 mg/m², 200 mg/m², 225 mg/m², 250 mg/m², 275 mg/m², 300 mg/m², 350 mg/m², 400 mg/m², 425 mg/m², 450 mg/m², 475 mg/m², 500 mg/m². Of course, all of these dosages are exemplary, and any dosage in-between these points is also expected to be of use in the invention.

iv. Bleomycin

Bleomycin is a mixture of cytotoxic glycopeptide antibiotics isolated from a strain of Streptomyces verticillus. Although the exact mechanism of action of bleomycin is unknown, available evidence would seem to indicate that the main mode of action is the inhibition of DNA synthesis with some evidence of lesser inhibition of RNA and protein synthesis.

In mice, high concentrations of bleomycin are found in the skin, lungs, kidneys, peritoneum, and lymphatics. Tumor cells of the skin and lungs have been found to have high concentrations of bleomycin in contrast to the low concentrations found in hematopoietic tissue. The low concentrations of bleomycin found in bone marrow may be related to.high levels of bleomycin degradative enzymes found in that tissue.

In patients with a creatinine clearance of >35 mL per minute, the serum or plasma terminal elimination half-life of bleomycin is approximately 115 minutes. In patients with a creatinine clearance of <35 mL per minute, the plasma or serum terminal elimination half-life increases exponentially as the creatinine clearance decreases. In humans, 60% to 70% of an administered dose is recovered in the urine as active bleomycin. Bleomycin may be given by the intramuscular, intravenous, or subcutaneous routes. It is freely soluble in water.

Bleomycin should be considered a palliative treatment. It has been shown to be useful in the management of the following neoplasms either as a single agent or in proven combinations with other approved chemotherapeutic agents in squamous cell carcinoma such as head and neck (including mouth, tongue, tonsil, nasopharynx, oropharynx, sinus, palate, lip, buccal mucosa, gingiva, epiglottis, larynx), skin, penis, cervix, and vulva. It has also been used in the treatment of lymphomas and testicular carcinoma. Because of the possibility of an anaphylactoid reaction, lymphoma patients should be treated with two units or less for the first two doses. If no acute reaction occurs, then the regular dosage schedule may be followed.

Improvement of Hodgkin's Disease and testicular tumors is prompt and noted within 2 weeks. If no improvement is seen by this time, improvement is unlikely. Squamous cell cancers respond more slowly, sometimes requiring as long as 3 weeks before any improvement is noted.

d. Corticosteroid Hormones

Corticosteroid hormones are useful in treating some types of cancer (lymphoma, leukemias, and multiple myeloma). Though these hormones have been used in the treatment of many non-cancer conditions, they are considered chemotherapy drugs when they are implemented to kill or slow the growth of cancer cells. Corticosteroid hormones can increase the effectiveness of other chemotherapy agents, and consequently, they are frequently used in combination treatments. Prednisone and dexamethasone are examples of corticosteroid hormones.

e. Mitotic Inhibitors

Mitotic inhibitors include plant alkaloids and other natural agents that can inhibit either protein synthesis required for cell division or mitosis. They operate during a specific phase during the cell cycle. Mitotic inhibitors comprise docetaxel, etoposide (VP16), paclitaxel, (TAXOL), vinblastine, vincristine, and vinorelbine.

i. Etoposide (VP16)

VP16 is also known as etoposide and is used primarily for treatment of testicular tumors, in combination with bleomycin and cisplatin, and in combination with cisplatin for small-cell carcinoma of the lung. It is also active against non-Hodgkin's lymphomas, acute nonlymphocytic leukemia, carcinoma of the breast, and Kaposi's sarcoma associated with acquired immunodeficiency syndrome (AIDS).

VP16 is available as a solution (20 mg/ml) for intravenous administration and as 50-mg, liquid-filled capsules for oral use. For small-cell carcinoma of the lung, the intravenous dose (in combination therapy) is can be as much as 100 mg/m² or as little as 2 mg/m², routinely 35 mg/m², daily for 4 days, to 50 mg/m², daily for 5 days have also been used. When given orally, the dose should be doubled. Hence the doses for small cell lung carcinoma may be as high as 200-250 mg/m². The intravenous dose for testicular cancer (in combination therapy) is 50 to 100 mg/m² daily for 5 days, or 100 mg/m² on alternate days, for three doses. Cycles of therapy are usually repeated every 3 to 4 weeks. The drug should be administered slowly during a 30- to 60-minute infusion in order to avoid hypotension and bronchospasm, which are probably due to the solvents used in the formulation.

ii. Taxol

TAXOL is an experimental antimitotic agent, isolated from the bark of the ash tree, Taxus brevifolia. It binds to tubulin (at a site distinct from that used by the vinca alkaloids) and promotes. the assembly of microtubules. TAXOL is currently being evaluated clinically; it has activity against malignant melanoma and carcinoma of the ovary. Maximal doses are 30 mg/m² per day for 5 days or 210 to 250 mg/m² given once every 3 weeks. Of course, all of these dosages are exemplary, and any dosage in-between these points is also expected to be of use in the invention.

iii. Vinblastine

Vinblastine is another example of a plant aklyloid that can be used in combination with gene therapy for the treatment of cancer and precancer. When cells are incubated with vinblastine, dissolution of the microtubules occurs.

Unpredictable absorption has been reported after oral administration of vinblastine or vincristine. At the usual clinical doses the peak concentration of each drug in plasma is approximately 0.4 mM. Vinblastine and vincristine bind to plasma proteins. They are extensively concentrated in platelets and to a lesser extent in leukocytes and erythrocytes.

After intravenous injection, vinblastine has a multiphasic pattern of clearance from the plasma; after distribution, the drug disappears from plasma with half-lives of approximately 1 and 20 hours. Vinblastine is metabolized in the liver to its biologically active derivative desacetylvinblastine. Approximately 15% of an administered dose is detected intact in the urine, and about 10% is recovered in the feces after biliary excretion. Doses should be reduced in patients with hepatic dysfunction. At least a 50% reduction in dosage is indicated if the concentration of bilirubin in plasma is greater than 3 mg/dl (about 50 mM).

Vinblastine sulfate is available in preparations for injection. The drug is given intravenously; special precautions must be taken against subcutaneous extravasation, since this may cause painful irritation and ulceration. The drug should not be injected into an extremity with impaired circulation. After a single dose of 0.3 mg/kg of body weight, myelosuppression reaches its maximum in 7 to 10 days. If a moderate level of leukopenia (approximately 3000 cells/mm³) is not attained, the weekly dose may be increased gradually by increments of 0.05 mg/kg of body weight. In regimens designed to cure testicular cancer, vinblastine is used in doses of 0.3 mg/kg every 3 weeks irrespective of blood cell counts or toxicity.

The most important clinical use of vinblastine is with bleomycin and cisplatin in the curative therapy of metastatic testicular tumors. Beneficial responses have been reported in various lymphomas, particularly Hodgkin's disease, where significant improvement may be noted in 50 to 90% of cases. The effectiveness of vinblastine in a high proportion of lymphomas is not diminished when the disease is refractory to alkylating agents. It is also active in Kaposi's sarcoma, neuroblastoma, and Letterer-Siwe disease (histiocytosis X), as well as in carcinoma of the breast and choriocarcinoma in women.

Doses of vinblastine will be determined by the clinician according to the individual patients need. 0.1 to 0.3 mg/kg can be administered or 1.5 to 2 mg/m² can also be administered. Alternatively, 0.1 mg/m² 0.12 mg/m², 0.14 mg/m², 0.15 mg/m², 0.2 mg/m², 0.25 mg/m², 0.5 mg/m², 1.0 mg/m², 1.2 mg/m², 1.4 mg/m², 1.5 mg/m², 2.0 mg/m², 2.5 mg/m², 5.0 mg/m², 6 mg/m², 8 mg/m², 9 mg/m², 10 mg/m², can be given. Of course, all of these dosages are exemplary, and any dosage in-between these points is also expected to be of use in the invention.

iv. Vincristine

Vincristine blocks mitosis and produces metaphase arrest. It seems likely that most of the biological activities of this drug can be explained by its ability to bind specifically to tubulin and to block the ability of protein to polymerize into microtubules. Through disruption of the microtubules of the mitotic apparatus, cell division is arrested in metaphase. The inability to segregate chromosomes correctly during mitosis presumably leads to cell death.

The relatively low toxicity of vincristine for normal marrow cells and epithelial cells make this agent unusual among anti-neoplastic drugs, and it is often included in combination with other myelosuppressive agents. Unpredictable absorption has been reported after oral administration of vinblastine or vincristine. At the usual clinical doses the peak concentration of each drug in plasma is approximately 0.4 mM.

Vinblastine and vincristine bind to plasma proteins. They are extensively concentrated in platelets and to a lesser extent in leukocytes and erythrocytes. Vincristine has a multiphasic pattern of clearance from the plasma; the terminal half-life is about 24 hours. The drug is metabolized in the liver, but no biologically active derivatives have been identified. Doses should be reduced in patients with hepatic dysfunction. At least a 50% reduction in dosage is indicated if the concentration of bilirubin in plasma is greater than 3 mg/dl (about 50 mM).

Vincristine sulfate is available as a solution (1 mg/ml) for intravenous injection. Vincristine used together with corticosteroids is presently the treatment of choice to induce remissions in childhood leukemia; the optimal dosages for these drugs appear to be vincristine, intravenously, 2 mg/mm² of body-surface area, weekly, and prednisone, orally, 40 mg/m², daily. Adult patients with Hodgkin's disease or non-Hodgkin's lymphomas usually receive vincristine as a part of a complex protocol. When used in the MOPP regimen, the recommended dose of vincristine is 1.4 mg/m². High doses of vincristine seem to be tolerated better by children with leukemia than by adults, who may experience sever neurological toxicity. Administration of the drug more frequently than every 7 days or at higher doses seems to increase the toxic manifestations without proportional improvement in the response rate. Precautions should also be used to avoid extravasation during intravenous administration of vincristine. Vincristine (and vinblastine) can be infused into the arterial blood supply of tumors in doses several times larger than those that can be administered intravenously with comparable toxicity.

Vincristine has been effective in Hodgkin's disease and other lymphomas. Although it appears to be somewhat less beneficial than vinblastine when used alone in Hodgkin's disease, when used with mechlorethamine, prednisone, and procarbazine (the so-called MOPP regimen), it is the preferred treatment for the advanced stages (III and IV) of this disease. In non-Hodgkin's lymphomas, vincristine is an important agent, particularly when used with cyclophosphamide, bleomycin, doxorubicin, and prednisone. Vincristine is more useful than vinblastine in lymphocytic leukemia. Beneficial response have been reported in patients with a variety of other neoplasms, particularly Wilms' tumor, neuroblastoma, brain tumors, rhabdomyosarcoma, and carcinomas of the breast, bladder, and the male and female reproductive systems.

Doses of vincristine for use will be determined by the clinician according to the individual patients need. 0.01 to 0.03 mg/kg or 0.4 to 1.4 mg/m² can be administered or 1.5 to 2 mg/m² can also be administered. Alternatively 0.02 mg/m², 0.05 mg/m², 0.06 mg/m², 0.07 mg/m², 0.08 mg/m², 0.1 mg/m², 0.12 mg/m², 0.14 mg/m², 0.15 mg/m², 0.25 mg/m² can be given as a constant intravenous infusion. Of course, all of these dosages are exemplary, and any dosage in-between these points is also expected to be of use in the invention.

f. Nitrosureas

Nitrosureas, like alkylating agents, inhibit DNA repair proteins. They are used to treat non-Hodgkin's lymphomas, multiple myeloma, malignant melanoma, in addition to brain tumors. Examples include carmustine and lomustine.

i. Carmustine

Carmustine (sterile carmustine) is one of the nitrosoureas used in the treatment of certain neoplastic diseases. It is 1,3bis (2-chloroethyl)-1-nitrosourea. It is lyophilized pale yellow flakes or congealed mass with a molecular weight of 214.06. It is highly soluble in alcohol and lipids, and poorly soluble in water. Carmustine is administered by intravenous infusion after reconstitution as recommended.

Although it is generally agreed that carmustine alkylates DNA and RNA, it is not cross resistant with other alkylators. As with other nitrosoureas, it may also inhibit several key enzymatic processes by carbamoylation of amino acids in proteins.

Carmustine is indicated as palliative therapy as a single agent or in established combination therapy with other approved chemotherapeutic agents in brain tumors such as glioblastoma, brainstem glioma, medullobladyoma, astrocytoma, ependymoma, and metastatic brain tumors. Also it has been used in combination with prednisone to treat multiple myeloma. Carmustine has proved useful, in the treatment of Hodgkin's Disease and in non-Hodgkin's lymphomas, as secondary therapy in combination with other approved drugs in patients who relapse while being treated with primary therapy, or who fail to respond to primary therapy.

Sterile carmustine is commonly available in 100 mg single dose vials of lyophilized material. The recommended dose of carmustine as a single agent in previously untreated patients is 150 to 200 mg/m² intravenously every 6 weeks. This may be given as a single dose or divided into daily injections such as 75 to 100 mg/m² on 2 successive days. When carmustine is used in combination with other myelosuppressive drugs or in patients in whom bone marrow reserve is depleted, the doses should be adjusted accordingly. Doses subsequent to the initial dose should be adjusted according to the hematologic response of the patient to the preceding dose. It is of course understood that other doses may be used in the present invention for example 10 mg/m², 20 mg/m², 30 mg/m², 40 mg/m², 50 mg/m², 60 mg/m², 70 mg/m², 80 mg/m², 90 mg/m², 100 mg/m². Some variation in dosage will necessarily occur depending on the condition of the subject being treated. The person responsible for administration will, in any event, determine the appropriate dose for the individual subject.

ii. Lomustine

Lomustine is one of the nitrosoureas used in the treatment of certain neoplastic diseases. It is 1-(2-chloro-ethyl)-3-cyclohexyl-1 nitrosourea. It is a yellow powder with the empirical formula of C₉H₁₆ClN₃O₂ and a molecular weight of 233.71. Lomustine is soluble in 10% ethanol (0.05 mg per mL) and in absolute alcohol (70 mg per mL). Lomustine is relatively insoluble in water (<0.05 mg per mL). It is relatively unionized at a physiological pH. Inactive ingredients in lomustine capsules are magnesium stearate and mannitol. Although it is generally agreed that lomustine alkylates DNA and RNA, it is not cross resistant with other alkylators. As with other nitrosoureas, it may also inhibit several key enzymatic processes by carbamoylation of amino acids in proteins.

Lomustine may be given orally. Following oral administration of radioactive lomustine at doses ranging from 30 mg/m² to 100 mg/m², about half of the radioactivity given was excreted in the form of degradation products within 24 hours. The serum half-life of the metabolites ranges from 16 hours to 2 days. Tissue levels are comparable to plasma levels at 15 minutes after intravenous administration.

Lomustine has been shown to be useful as a single agent in addition to other treatment modalities, or in established combination therapy with other approved chemotherapeutic agents in both primary and metastatic brain tumors, in patients who have already received appropriate surgical and/or radiotherapeutic procedures. It has also proved effective in secondary therapy against Hodgkin's Disease in combination with other approved drugs in patients who relapse while being treated with primary therapy, or who fail to respond to primary therapy.

The recommended dose of lomustine in adults and children as a single agent in previously untreated patients is 130 mg/m² as a single oral dose every 6. weeks. In individuals with compromised bone marrow function, the dose should be reduced to 100 mg/m² every 6 weeks. When lomustine is used in combination with other myelosuppressive drugs, the doses should be adjusted accordingly. It is understood that other doses may be used for example, 20 mg/m² 30 mg/m², 40 mg/m², 50 mg/m², 60 mg/m², 70 mg/m², 80 mg/m2, 90 mg/m², 100 mg/m², 120 mg/m² or any doses between these figures as determined by the clinician to be necessary for the individual being treated.

g. Miscellaneous Chemotherapuetic Agents

Some chemotherapy agents do not qualify to be listed into the previous categories based on their activities. However, it is contemplated that they are included within the method of the present invention for use in combination therapies of cancer with gene therapy involving lipid formulations. They include amsacrine, L-asparaginase, and tretinoin.

2. Radiotherapy

Another therapy that may be used in conjunction with the therapeutic composition of the present invention to treat a cancer is radiotherapy. It is contemplated that radiotherapeutic factors that may be employed in the present invention are factors that cause DNA damage and have been used extensively, such as y-rays, X-rays, and/or the direct delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the cancer or tumor cells.

3. Immunotherapy

The present invention also contemplates the use of immunotherapy in conjunction with the therapeutic composition. Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy could also be used as part of a combined therapy. The general approach for combined therapy is discussed herein. In one aspect of immunotherapy, the tumor cell must bear some marker that is amenable to targeting, i.e., is not present on the majority of other cells. Many tumor markers exist and any of these may be suitable for targeting in the context of the present invention. Common tumor markers which have been found to be upregulated in various cancers include, but are not limited to carcinoembryonic antigen, CA 27-29 antigen, neuron-specific enolase (NSE), CA 125 antigen, and human chorionic gonadotropin (HCG). An alternative aspect of immunotherapy is to anticancer effects with immune stimulatory effects.

Other types of immunotherapy that may be employed with the therapeutic composition of the present invention are passive and active immunotherapy.

A number of different approaches for passive immunotherapy of cancer exist. They may be broadly categorized into the following: injection of antibodies alone; injection of antibodies coupled to toxins or chemotherapeutic agents; injection of antibodies coupled to radioactive isotopes; injection of anti-idiotype antibodies; and finally, purging of tumor cells in bone marrow. It may be favorable to administer more than one monoclonal antibody directed against two different antigens or even antibodies with multiple antigen specificity. Treatment protocols also may include administration of lymphokines or other immune enhancers as described by Bajorin et al. (1988). The development of human monoclonal antibodies is well known to those of skill in the art (see Harlow and Lane, 1988).

In active immunotherapy, an antigenic peptide, polypeptide or protein, or an autologous or allogenic tumor cell composition or “vaccine” is administered, generally with a distinct bacterial adjuvant (Ravindranath and Morton, 1991; Mitchell et al., 1990; Mitchell et al., 1993).

4. Seconary Gene Therapy

The present invention also contemplates adjunct gene therapy in conjunction with the therapeutic composition. As with the majority of human cancers, numerous genetic alterations have been identified that play a role in adenocarcinomas. These include mutations in the tumor suppressor genes p53, Rb, p16, BRCA2 and DPC4. Several activated oncogenes have also been identified as contributing to cancers including K-ras, HER-2/neu, NFkappaB and AKT2. There are, no doubt, many other genetic defects that contribute to the onset and progression of cancer and identifying these mutants and the specific consequences of the defects will lead to a better understanding of how to treat this disease. Thus, the present invention contemplates using a second tumor therapeutic gene from those discussed in Section A, above.

5. Hormonal Therapy

Hormonal therapy may also be used in conjunction with therapeutic composition of the present invention or in combination with any other cancer therapy described herein. The use of hormones may be employed to lower the level or block the effects of certain hormones that may play a role in the tumor cell proliferation. This treatment is often used in combination with at least one other cancer therapy as a treatment option or to reduce the risk of metastases in cancers which include, but are not limited to, breast, prostate, ovarian, or cervical cancer.

6. Surgery

The present invention may also be used in conjunction with surgery. Surgery may also be used in combination with any of the other cancer therapies described herein such as radiation therapy.

I. Pharmaceutical Compositions

In a particular aspect, the present invention provides methods for inhibiting tumor and the treatment of related diseases such as cancers or hyperproliferative diseases. Treatment methods will involve treating an individual with an effective amount of a composition comprising a tumor therapeutic expression construct and doxorubicin, cyclophosphamide, 5-FU, taxol or gemcitabine. Such compositions may be provided as isolated and substantially purified protein in pharmaceutically acceptable formulations using formulation methods known to those of ordinary skill in the art. These formulations can be administered by standard routes. In general, the combinations may be administered by intratumoral, parenteral (e.g., intravenous, subcutaneous or intramuscular), topical, transdermal, direct, intraperitoneal, oral, rectal or administration, but is not limited to such. In addition, a composition comprising tumor therapeutic expression construct and doxorubicin, cyclophosphamide, 5-FU, taxol or gemcitabine may be incorporated into biodegradable polymers allowing for sustained release of the compound, the polymers being implanted in the vicinity of where drug delivery is desired, for example, at the site of a tumor or implanted so that the composition is slowly released systemically.

In an alternative embodiment, a composition comprising a tumor therapeutic expression construct and doxorubicin, cyclophosphamide, 5-FU, taxol or gemcitabine may be provided as a protein composition for example in an aqueous solution or as a liposomal complex. Liposomes as delivery vehicles presented above for nucleic acid constructs is equally applicable to delivery of protein or other drug compositions. Further, a composition comprising a tumor therapeutic expression construct and doxorubicin, cyclophosphamide, 5-FU, taxol or gemcitabine may be provided as described above in a viral expression construct containing a gene that encodes a tumor therapeutic. An effective amount is described, generally, as that amount sufficient to detectably and repeatedly ameliorate, reduce, minimize or limit the extent of a disease or its symptoms. More rigorous definitions may apply, including elimination, eradication or cure of disease. In the context of the present invention, the diseases include cancers, and extend into affecting conditions that alter the progression of the disease, for example, angiogenesis and/or the effect of inhibiting angiogenesis on tumor growth.

Where clinical application of a gene therapy is contemplated, it will be necessary to prepare the complex as a pharmaceutical composition appropriate for the intended application. Generally this will entail preparing a pharmaceutical composition that is essentially free of pyrogens, as well as any other impurities that could be harmful to humans or animals. One also will generally desire to employ appropriate salts and buffers to render the complex stable and allow for complex uptake by target cells.

Aqueous compositions of the present invention comprise an effective amount of the compounds, dissolved or dispersed in a pharmaceutically acceptable carrier or aqueous medium. Such compositions can also be referred to as inocula. The phrases “pharmaceutically or pharmacologically acceptable” refer to molecular entities and compositions that do not produce an adverse, allergic or other untoward reaction when administered to an animal, or a human, as appropriate. As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients also can be incorporated into the compositions.

The compositions of the present invention may include classic pharmaceutical preparations. Dispersions also can be prepared in glycerol, liquid polyethylene glycols, and mixtures thereof and in oils. Under ordinary conditions of storage and use, these preparations contain a preservative to prevent the growth of microorganisms.

Depending on the particular cancer to be treated, administration of therapeutic compositions according to the present invention will be via any common route so long as the target tissue is available via that route. This includes oral, nasal, buccal, rectal, vaginal or topical. Topical administration would be particularly advantageous for treatment of skin cancers. Alternatively, administration may be by orthotopic, intradermal, subcutaneous, intramuscular, intraperitoneal or intravenous injection. Such compositions would normally be administered as pharmaceutically acceptable compositions that include physiologically acceptable carriers, buffers or other excipients.

The therapeutic compositions of the present invention are advantageously administered in the form of injectable compositions either as liquid solutions or suspensions; solid forms suitable for solution in, or suspension in, liquid prior to injection may also be prepared. These preparations also may be emulsified. A typical composition for such purpose comprises a pharmaceutically acceptable carrier. For instance, the composition may contain 10 mg, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like. Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH and exact concentration of the various components the pharmaceutical composition are adjusted according to well known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders. When the route is topical, the form may be a cream, ointment, salve or spray.

### J. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Cells and cell culture. PC-3, a human prostate adenocarcinoma cell line, was obtained from the American Type Culture Collection and DHD/K12/TRb (PROb), a rat colon adenocarcinoma established in syngeneic BD-IX rats by 1,2-dimethylhydrazine induction (obtained from Francois Martin, University of Dijon, France) were used. The human prostate carcinoma cell line PC-3 (American Type Culture Collection, Manassas, Va.) was maintained in DMEM-F12 (Invitrogen Life Technologies, Carlsbad, Calif.) supplemented with FBS (10% vol/vol) (Intergen, Purchase, N.Y.), penicillin (100 IU/ml), and streptomycin (100 tg/ml) (Invitrogen Life Technologies) at 37° C. with 7.5% CO₂ PC-3 cells are p53 null (Cemazar et al., 2003) express Pgp, MRP, GST-π (van Brussel et al., 1999) and Bcl-2. (Sinha et al., 1995). The rat colon adenocarcinoma cell line DHD/K12/TRb(PROb) was obtained from Francois Martin (University of Dijon, France), and was established in BD-IX rats by injection of 1,2-dimethylhydrazine. PROb cells were maintained in DMEM (Invitrogen Life Technologies) supplemented with FBS (10% vol/vol) (Intergen), penicillin (100 IU/ml), and streptomycin (100 μg/ml) (Invitrogen Life Technologies) at 37° C. with 7.5% CO₂. There is little published information on the molecular/genetic characteristics of PROb cells.

Chemical reagents. N-Acetylcysteine (NAC) was obtained from Roxane Laboratories, Inc. (Columbus, Ohio). Cisplatin and fluorouracil were obtained from American Pharmaceutical Partners (Schaumburg, Ill.). Doxorubicin was manufactured by Ben Venue Laboratories (Bedford, Ohio). Gemcitabine was obtained from Eli Lilly (Indianapolis, Ind.). Paclitaxel was manufactured by F. H. Faulding (Mulgrave Victoria, Australia). Cyclophosphamide was obtained from Bristol-Myers Squibb (Princeton, N.J.).

Animals. The in vivo experiments were conducted using female athymic nude mice.

Xenografts. PC-3 xenografts were established by injection of 10⁷ cells in 100 μl of PBS into the right hind limb of 6-week old female athymic nude mice (Frederick Cancer Research Institute, Frederick, Md.). PROb xenografts were established by injecting 5×10⁶ cells in 100 μl of PBS. Experiments were conducted 2-3 weeks after injection when tumors reached an average size of 200-300 mm³. Experiments were conducted in accordance with the guidelines of the Institutional Animal Care and Use Committee of the University of Chicago.

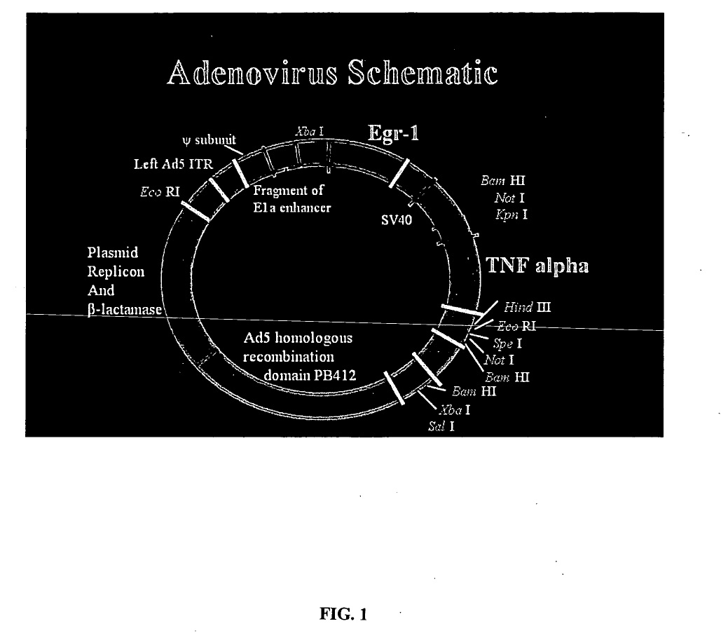

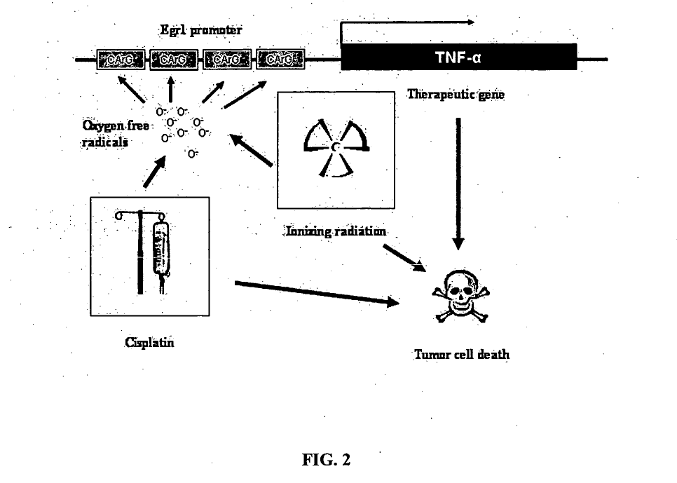

Viral vectors. Ad.Egr.TNF.11D (GenVec Inc., Gaithersburg, Md.), a replication-deficient adenoviral vector (E1-, partially E3-, E4- deleted) containing the human TNF-α gene under the control of the radiation-inducible promoter Egr-1, was stored at −80° C. and was diluted in formulation buffer (GenVec) to the appropriate concentration. See FIG. 1 and FIG. 2.

In vitro measurement of TNF-α protein. PC-3 and PROb cells were plated in 96-well plates, grown overnight, and infected with Ad.Egr.TNF at 100 multiplicities of infection in serum-free. Cisplatin (250 μM), doxorubicin (3 μM), 5-fluorouracil (20 mM) and paclitaxel (14 μM), were added in serum-containing media after incubation for 3 hours. Supernatants were harvested 24 hrs later and human TNF-α production was quantified by ELISA. These experiments were performed in quintuplicate. Data are expressed as mean±SD.

In vivo measurement of TNF-α protein. PC-3 (1×10⁷ cells) or PROb cells (5×10⁶) in 100 μl were injected subcutaneously into the right hind limb of nude mice. Tumor-bearing mice were randomized to normal saline (NS) as control or one chemotherapeutic agent: cisplatin (9 mg/kg), cyclophosphamide (160 mg/kg), doxorubicin (15 mg/kg), 5-fluorouracil (100 mg/kg) and ge mcitabine (500 mg/kg). Each mouse received intratumoral Ad.Egr.TNF (5×10⁹ particle units [p.u.] in 10 μl) with 250 μl of complete media with normal saline or a chemotherapeutic agent. IP injections were administered 20 hrs after transfection, and two consecutive IT and IP injections were given. Animals were euthanized, and xenografts were harvested 48 hrs after the second IP injection. Xenografts were snap-frozen in liquid nitrogen and homogenized in RIPA buffer (150 mM NaCl, 10 mM Tris at pH 7.5, 5 mM EDTA at pH 7.5, 100 mM PMSF, 1 μg/ml leupeptin, and 2 μg/ml aprotinin). After three freeze-thaw lysis cycles, the homogenate was centrifuged for 10 minutes at 4° C. TNF-α levels in the supernatants were measured as described above.

Efficacy study. PC-3 xenografts were established as above. On day 0, tumor-bearing mice were volume-matched and assigned to one of four groups: normal saline as control, Ad.Egr.TNF only (5×10⁹ p.u. in 10 μl intraperitonealy on days 0 and 3), doxorubicin (2 mg/kg ip daily for 10 days), or a combined treatment of Ad.Egr.TNF and doxorubicin. Xenografts volumes (length×width×thickness/2) were measured using calipers twice weekly. Fractional tumor volumes (V/Vo where Vo=volume on day 0) were calculated and plotted. Day 0 is the day of randomization and the first day of treatment.

Chemo-sensitivity of PC-3 and PROb cells as determined by MTS assay. PC-3 and PROb cells were plated at a density of 10⁵ cells in 100 μl of medium per well in flat-bottom 96-well tissue culture plates and incubated overnight. The medium was removed, and cells were infected with Ad.Egr.TNF.11D in serum-free medium at 0 and 100 multiplicities of infection (MOI) for 3 h. After incubation, 200 μl of complete media with or without chemotherapeutic agents was added. Chemotherapeutic agents used were at final concentrations of cisplatin at 46 and 460 μM; doxorubicin at 3 and 300 μM; 5-fluorouracil at 2 and 200 mM; and paclitaxel at 1.4 and 140 μM. Media was removed 24 h later and each well was rinsed with 200 μl of complete media (CM) and aspirated. 100 μl of CM was then added with 20 μl of CellTiter 96® AQᵤₑₒᵤₛ One Solution Cell Proliferation Assay solution (MTS assay; Promega, Madison, Wis.). Cells were then allowed to incubate for 1 h. Absorbance was measured at 490-650 nm.

Chemo-inducibility of Ad.Egr-TNF.11D in vitro. PC-3 and PROb cells were plated at a density of 10⁵ cells in 100 μl of CM per well in flat-bottom 96-well tissue culture plates and incubated overnight. The medium was removed, and cells were infected with Ad.Egr.TNF.11D (GenVec) at 0 and 100 MOI in 100 μl serum-free medium for 3 h. After incubation, 200 μl of CM with or without chemotherapeutic agents was added. The chemotherapeutic agents used were at a final concentration of cisplatin 250 μM, doxorubicin at 3 μM, 5-fluorouracil at 100 mM, gemcitabine at 3 mM and paclitaxel at 14 μM. Conditioned medium was harvested 24 h later, and TNF-α concentration was measured using a Quantikine Human TNF-α ELISA kit (R & D Systems, Minneapolis, Minn.).

Chemo-inducibility of Ad.Egr-TNF.11D in vivo. PC-3 and PROb xenografts were injected intratumorally (i.t.) with 5×10⁹ particle units (p.u.) of Ad.Egr.TNF.11D on days 0 and 1. Chemotherapeutic agents administered intraperitoneally (i.p.) on days 1 and 2, included cisplatin (9 mg/kg), cyclophosphamide (160 mg/kg), doxorubicin (15 mg/kg), 5-fluorouracil (100 mg/kg) and gemcitabine (500 mg/kg). The control group received normal saline. Animals were euthanized, and xenografts were harvested 24 h after the second i.p. injection. Xenografts were snap-frozen in liquid nitrogen and homogenized in RIPA buffer (150 mM NaCl, 10 mM Tris pH 7.5, 5 mM EDTA pH 7.5, 100 mM PMSF, 1 μg/ml leupeptin, and 2 μg/ml aprotinin) using a Brinkman Polytron homogenizer (Kinematica AG, Lucerne, Switzerland). After three freeze-thaw lysis cycles, the homogenate was centrifuged at 7800×g in a Sorvall RC-5C SS34 rotor (Kendro Laboratory Products, Newtown, Conn.) for 10 min at 4° C. TNF-α levels in the supernatants were measured by ELISA as described above.

N-acetyl cysteine effects on TNF-60 production in vitro. PC-3 and PROb cells were plated and infected with Ad.Egr-TNF.11D as described above. PC-3 and PROb cells were treated with N-acetylcysteine (NAC) at 0 mM, 10 mM, 20 mM and 30 mM, followed immediately by the addition of 100 mM 5-FU. Conditioned medium was collected after 24 h of incubation at 37° C. and stored at −20° C. TNF-α levels were determined by ELISA.

PC-3 and PROb cells were plated and infected with Ad.Egr-TNF.11D as above. Prior to the addit ion of chemotherapeutic agents (cisplatin, doxorubicin, 5-FU, gemcitabine and paclitaxel), 20 mM NAC in 0.1 ml complete medium was added to each well. Conditioned medium was collected after 24 h of incubation at 37° C., and stored at −20° C. TNF-α levels were determined by ELISA.

Xenograft regrowth studies. PC-3 and PROb xenografts were established in nude mice as described, (Chung et al., 1998; Park et al., 2002). Treatment was initiated on day 0 at which time mice were assigned to one of 4 treatment groups: control, doxorubicin, Ad.Egr-TNF.11D, and combination of Ad.Egr-TNF.11D and doxorubicin. On days 0 and 3, mice received intratumoral (i.t.) injection of 10 μl of either 5×10⁹ p.u. Ad.Egr-TNF.11D (vector alone and combination groups), or 10 μl of viral formulation buffer (control and doxorubicin groups). Intraperitoneal (i.p.) injections of doxorubicin (2 mg/kg) or an equal volume of normal saline were administered daily from days 0 through 8. Xenografts were measured twice weekly and tumor volume was calculated according to the formula (length×width×thickness)/2. (Hallahan et al., 1995). Fractional tumor volumes (V/V₀ where V₀=volume on day 0) were calculated and plotted.

Analysis of microvessel density. Two or three xenografts from each treatment group in the PC-3 regrowth study above, including control, doxorubicin alone, Ad.Egr-TNF.11D alone and the combination of Ad.Egr-TNF.11D plus doxorubicin, were collected and fixed in 10% neutral buffered formalin. Tissues were embedded in paraffin, cut in 5 μm slices, mounted, baked, cleared in xylene, and rehydrated in decreasing alcohol concentrations (100%-70%) and distilled water. Sections were microwaved in 10 mM citrate buffer, pH 6.0, for 18 min, washed and soaked in 1% hydrogen peroxide/methanol for 20 min prior to blocking with avidin-biotin (Vector Laboratories, Burlingame, Calif.) for 15 min. Slides were incubated with biotin (15 min), washed and blocked with serum-free DAKO protein (DAKO, Carpinteria, Calif.) for 10 min prior to incubation with a 1:50 dilution of goat anti-mouse CD31 antibody (Santa Cruz, Santa Cruz, Calif.) for 60 min at RT. CD-31 staining was visualized on tissue sections following incubation with DAKO biotinylated anti-goat secondary antibody for 30 min and DAB reagent (Vector) for 60 sec. Sections were counterstained with Gill 3 hematoxylin, dehydrated in ethanol (95%-100%) and xylene prior to mounting. All slides were read by an investigator blinded to the treatment groups. Positively stained vessels were counted in 5-10 high power fields (x400) per slide using light microscopy. Blood vessels were identified by endothelial cell staining and by endothelial cells surrounding intraluminal erythrocytes.

Statistical analysis. Statistical significance was determined by one-way analysis of variance (ANOVA). Differences between treatment groups were determined by either student's t test or Mann-Whitney rank sum test.

### Example 2

### Preliminary Results

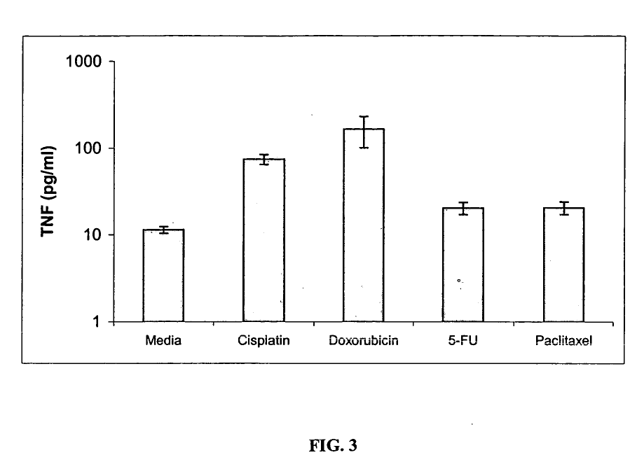

In vitro. PC-3 cells treated with Ad.TNF showed increased TNF levels with the addition of cisplatin (6.5-fold, p<0.001), doxorubicin (14.5, p<0.001), 5-fluorouracil (1.8-fold, p<0.001) and paclitaxel (1.8-fold, p<0.001). These results are shown in FIG. 3.

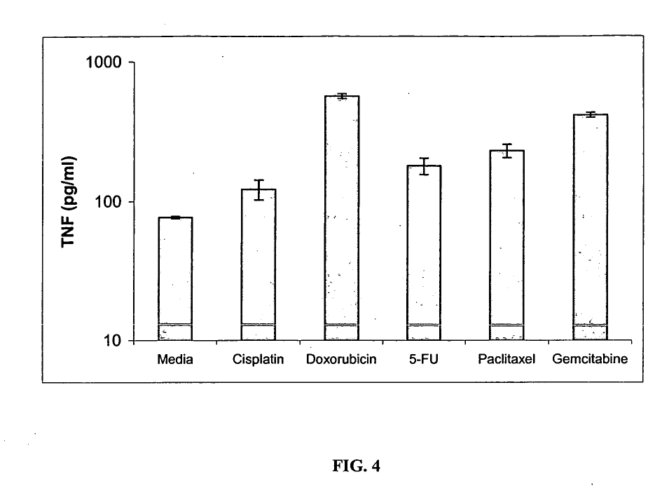

PROb cells treated with Ad.TNF showed increased TNF levels with the addition of cisplatin (1.6-fold, p<0.001), doxorubicin (7.3-fold, p<0.001), 5-fluorouracil (2.3-fold, p<0.001) and paclitaxel (3.0-fold, p<0.001), and gemcitabine (5.4-fold, p<0.001). These results are shown in FIG. 4).

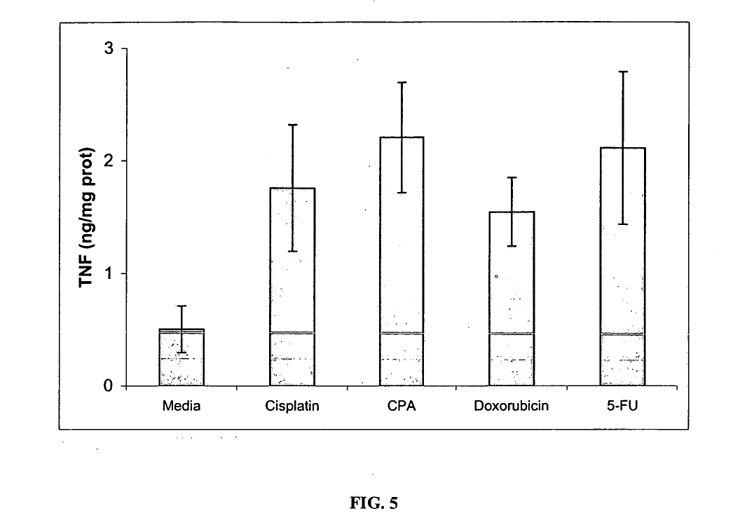

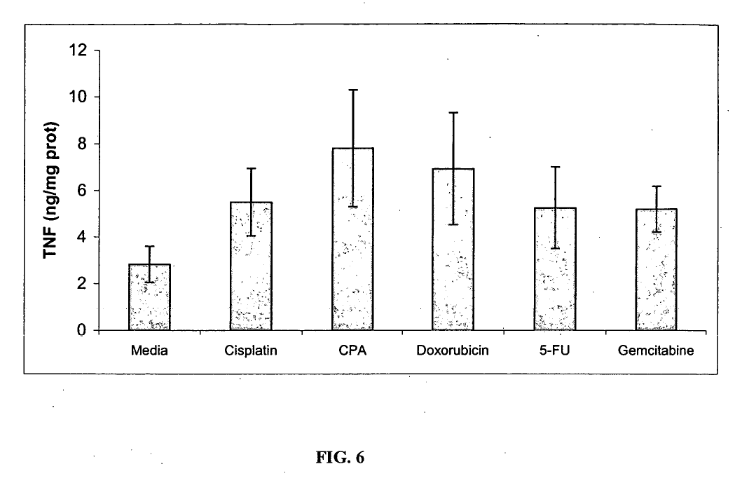

In vivo study. In PC-3 xenografts, agents that induced TNF-α were cisplatin (3.5-fold, p=0.08), cyclophosphamide (4.4-fold, p=0.01), doxorubicin (3.1-fold, p=0.04) and 5-fluorouracil (4.2-fold, p=0.08), when compared with mice treated with vector and NS. These results are shown in FIG. 5. PROb cells treated with Ad.TNF demonstrated increased TNF-α levels when given concurrently with cisplatin (1.9-fold, p=0.12), cyclophosphamide (2.8 (p=0.06), doxorubicin (2.5-fold, p=0. 19), 5-fluorouracil (1.9-fold, p=0.31) and gemcitabine (1.8-fold, p=0.17) compared with cells exposed to vector plus normal saline. These results are shown in FIG. 6.

Efficacy study. Tumor volumes of the mice treated with doxorubicin were similar to the control group. Ad.Egr.TNF treatment alone resulted in some growth delay; however, the combined treatment with Ad.Egr.TNF and doxorubicin resulted in a decrease of tumor volumes when compared to control (p=0.02), doxorubicin alone (p<0.001) and Ad.Egr.TNF alone (p=0.07). These results are shown in FIG. 7.

### Example 3

### Chemo-sensitivity of PC-3 and PROb Cells

Percent survival following exposure to Ad.Egr-TNF.11D and chemotherapy was compared with survival in growth media. PC-3 cells demonstrated surviving fractions of 60% (460 μM) and 90% (46 μM) with cisplatin; 30% (300 μM) and 90% (3 μM) with doxorubicin; 20% (200 mM) and 80% (2 mM) with 5-FUl and 10% (140 μM) and 80% (1.4 μM) with taxol. PROb demonstrated surviving fractions of 77% (460 μM) and 77% (46 μM) with cisplatin; 85% (300 μM) and 100% (3 μM) with doxorubicin; 38% (200 mM) and 69% (2 mM) with 5-FU; and 8% (140 μM) and. 85% (1.4 μM) with taxol.

### Example 4

### Induction of TNF-α Protein

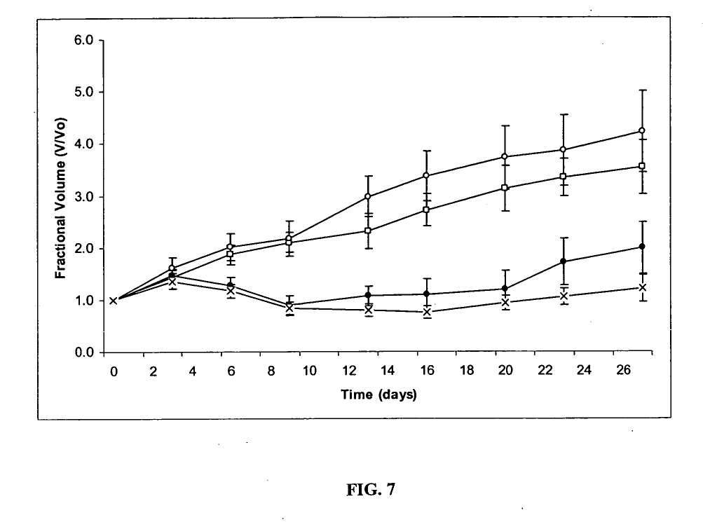

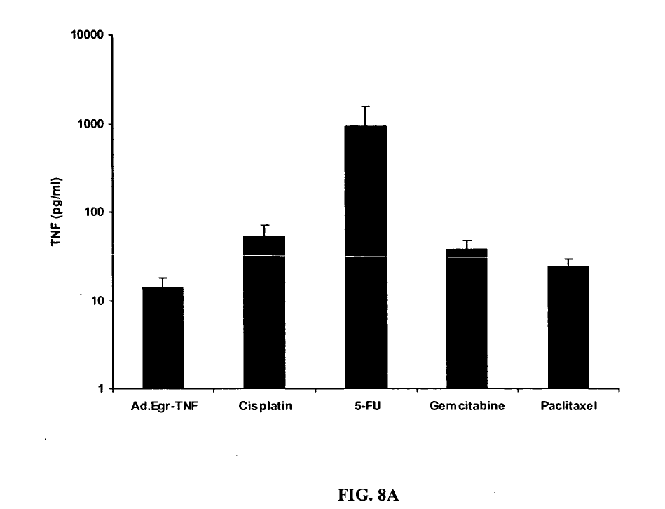

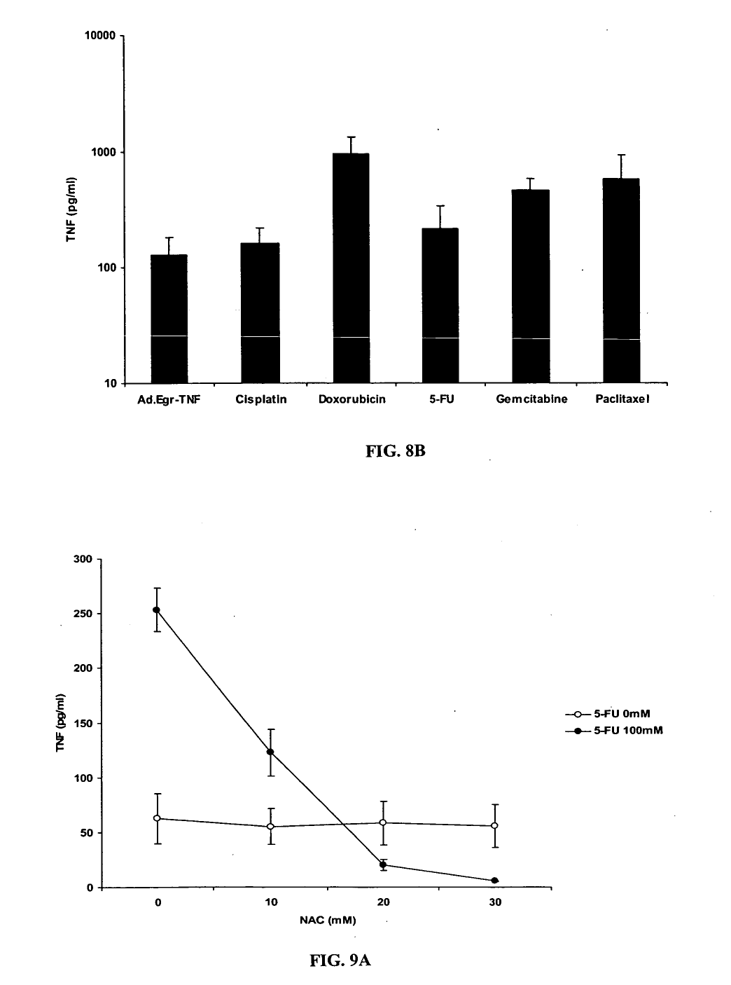

Using an ELISA specific for human TNF-α, TNF-α production was assessed following infection of PC-3 cells and PROb cells with 100 MOI of Ad.Egr-TNF.11D. Neither of these cell lines produced endogenous human TNF-α. Following infection with Ad.Egr-TNF.11D, PC-3 cells produce 14 pg/ml of TNF-α and PROb cells produce 130 pg/ml. Next, PC-3 and PROb cells were infected with Ad.Egr-TNF.11D and exposed to cisplatin (250 μM), doxorubicin (3 μM), 5-FU (100 mM), gemcitabine (3 mM) or paclitaxel (14 μM), based on LD₅₀ values on a panel of human tumor cell lines obtained from the National Institutes of Health (NIH) website located on the internet. Induction of TNF-α by cyclophosphamide was not investigated in vitro because this drug requires hepatic activation. In PC-3 cells infected with Ad.Egr-TNF.11D, significant increases in TNF-α levels were detected following exposure to cisplatin (3.8-fold increase), 5-FU (67.4-fold increase), gemcitabine (2.7-fold increase), and paclitaxel (1.7-fold increase, p<0.001, FIG. 8A). Induction of TNF-α by doxorubicin was not evaluated because doxorubicin was toxic to PC-3 cells at the doses used in these experiments. Similar results were obtained using PROb cells infected with Ad.Egr-TNF.11D. Significant increases in TNF-α levels were found following exposure to cisplatin (1.3-fold increase, p=0.04), 5-FU (1.7-fold increase, p<0.02), gemcitabine (3.5-fold increase, p<0.001), and paclitaxel (4.5-fold increase, p<0.001, FIG. 8B). The greatest induction of TNF-α in PROb cells was observed following infection with Ad.Egr-TNF.11D and exposure to doxorubicin (7.4-fold increase, p<0.001). These data obtained from histologically different cancer cell lin es demonstrate that Ad.Egr-TNF.11D is activated by different classes of chemotherapeutic agents.

### Example 5

### N-ACETYL Cysteine Alters Induction of TNF-α Protein

Based on previous studies that demonstrated transcriptional activation of the Egr-1 promoter through the CArG sequences by IR-mediated ROIs (Hallahan et al., 1991; Nose et al., 1991; Datta et al., 1992; Datta et al., 1993), it was hypothesized that chemotherapeutic agents reported to induce intracellular ROIs would also activate Ad.Egr-TNF.11D and produce therapeutic levels of TNF-α protein. Notably, cisplatin, (Sodhi and Gupta, 1986; Senturker et al., 2002), cyclophosphamide (Sulkowska et al., 2003), doxorubicin, 5-FU (Ueta et al., 1999), gemcitabine (van der Donk et al., 1998) and paclitaxel (Varbiro et al., 2001) have been reported to induce intracellular ROIs and/or intracellular changes in redox potential. Consequently, the inventors tested whether NAC, a free radical scavenger, would decrease TNF-α production if present at the time of addition of chemotherapeutic agents.

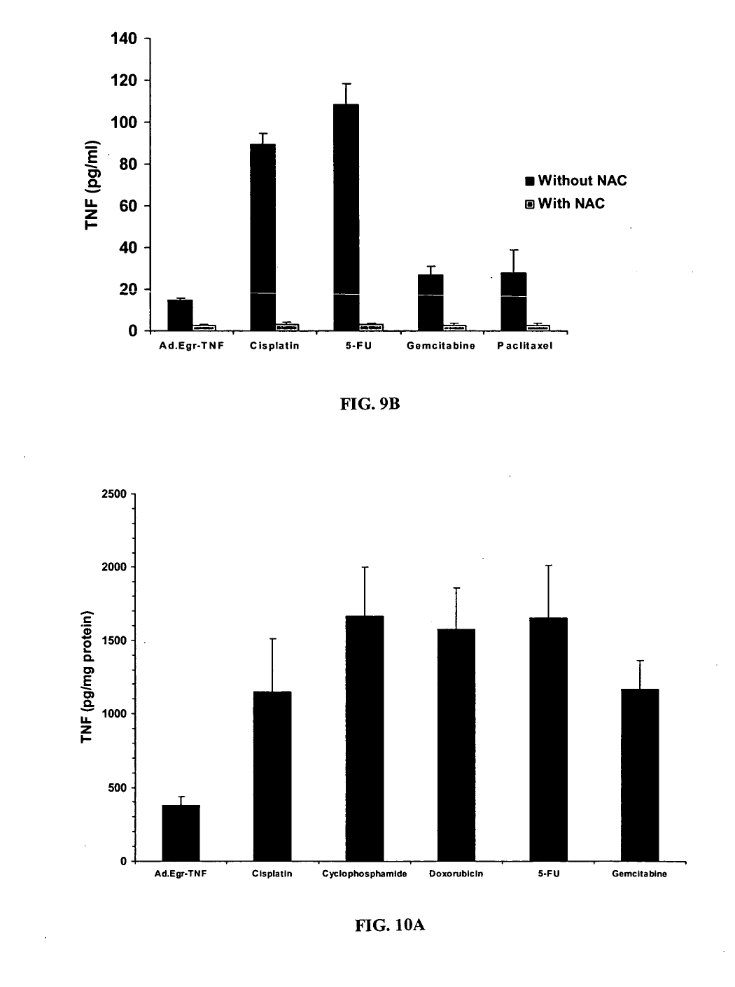

First, the effect of NAC on TNF-α production following exposure to 5-FU was examined. FIG. 9A shows that increasing concentrations of NAC (from 10 mM to 30 mM), decrease the concentration of TNF-α protein produced by PC-3 cells infected with Ad.Egr-TNF.11 D and treated with 100 mM 5-FU compared with PC-3 cells infected with Ad.Egr-TNF.11 D alone. Next the effect of NAC on TNF-60 induction by the same panel of chemotherapeutic agents used in the in vitro chemo-induction experiments was investigated. NAC significantly decreased the concentration of TNF-α protein produced by Ad.Egr-TNF.11 D transduced PC-3 cells treated with cisplatin, 5-FU, gemcitabine and paclitaxel (p<0.042, FIG. 9B). The induction of TNF-α following treatment with 3 μM doxorubicin in PROb cells was significantly reduced (p<0.001) in the presence of NAC. Similar results were obtained when PROb cells were treated with cisplatin, 5-FU, gemcitabine or paclitaxel and exposed to NAC (data not shown). The activation of Ad.Egr-TNF.11D by all of the chemotherapeutic compounds studied in the present work was altered by NAC.

Taken together, these data demonstrate that activation of the Egr-TNF construct is mediated, at least in large part, by ROIs produced by these chemotherapeutic agents. The induction of Egr-1 by agents that produce ROIs is consistent with reports that changes in cellular oxidation/reduction regulate the activation of several transcription factors including c-Fos and c-Jun, (Abate et al., 1990; Nose et al., 1991; Zafarullah et al., 2003; Li et al., 1994). Mitomycin C, vincristine, topotecan, resveratrol and cisplatin have also been shown to activate egr-1 transcription (Quinones et al., 2003; Park et al., 2002). The available data on egr-1 gene induction, considered together with the results reported herein, suggest that chemo-inducible gene therapy based on control of transgene expression by free radical production may be applicable to diverse chemotherapeutic agents. It is noteworthy that several studies report constitutive activity of the Egr-1 promoter. Although, low levels of TNF-α are produced by the Ad.Egr-TNF vector, toxicity has not been observed in animal or human studies.

### Example 6

### In vivo Induction of TNF-α Protein

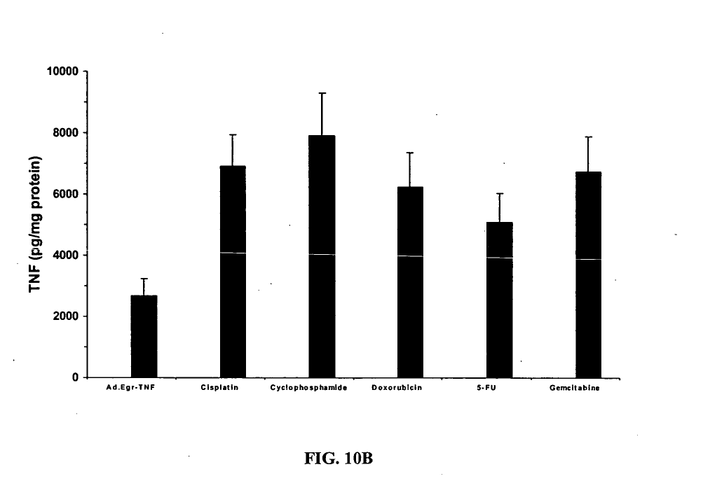

Next the induction of human TNF-α by chemotherapeutic agents in PC-3 and PROb tumors growing in nude mice was investigated. Xenografts were injected with Ad.Egr-TNF.11D on days 0 and 1, and chemotherapy was administered on days 1 and 2. Significant increases in human TNF-α levels in the tumors were detected 48 h after the second injection of Ad.Egr-TNF.11D. PC-3 tumors injected with Ad.Egr-TNF.11D alone produced 376.33±64.22 pg/mg of TNF-α protein. The combination of Ad.Egr-TNF.11D and chemotherapy produced a significant increase in TNF-α levels following treatment with cisplatin (3.1-fold increase, p=0.062), cyclophosphamide (4.4-fold increase, p<0.001), doxorubicin (4.2-fold increase, p<0.001), 5-FU (4.4-fold increase, p<0.001), and gemcitabine (3.1-fold increase, p<0.001, FIG. 10A). In PROb xenografts, significant induction of TNF-α protein was detected following combined treatment with Ad.Egr-TNF.11D and cisplatin (2.6-fold increase, p=0.002), cyclophosphamide (3.0-fold increase, p<0.001), doxorubicin (2.3-fold increase, p<0.001), 5-FU (1.9-fold increase, p=0.023), and gemcitabine (2.5-fold increase, p<0.001) compared to treatment with Ad.Egr-TNF.11D alone (FIG. 10B). Studies of in vivo induction by taxol were not feasible due to severe systemic toxicity at the doses employed in these studies. The results demonstrate that, like IR, chemotherapeutic agents induce the production of TNF-α protein by tumors transduced with the Ad.Egr-TNF.11D vector.

### Example 7

### Xenografts Regrowth Studies

PC-3 tumors have been shown to be resistant to doxorubicin in vivo (Teicher et al., 1997) and PC-3 cells resistant to TNF-α in vitro (data not shown). Based on previous studies demonstrating that radio-induction of Ad.Egr-TNF.11D produces significant anti-tumor effects in radioresistant tumors due to the destruction of the tumor microvasculature, (Hallahan et al., 1995; Staba et al., 1998; Mauceri et al., 1996), it was determined whether the combination of Ad.Egr-TNF.11D and doxorubicin would be effective in overcoming resistance to chemotherapy and/or TNF-α.

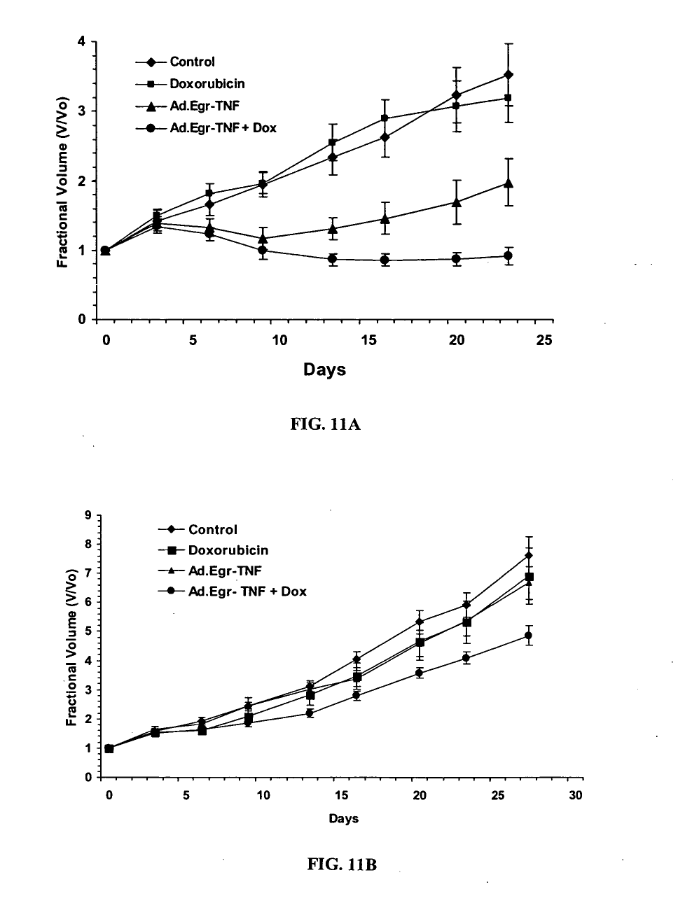

PC-3 tumors (initial mean tumor volume=368±22 mm³, n=59) were injected with Ad.Egr-TNF.11D and mice were treated with doxorubicin. The data obtained from two independent experiments were combined and are shown in FIGS. 11A-B. Mice in the control group (injected i.t. with viral buffer and i.p. with saline) and those in doxorubicin group (injected i.t. with viral buffer and i.p. with doxorubicin) exhibited equivalent tumor growth with mean volume increasing by 3-fold at day 23. Treatment with Ad.Egr-TNF.11D alone significantly reduced mean tumor volume beginning on day 9 (p=0.008) and continuing to day 23 (p=0.005) compared to the buffer injected control group. The combination of Ad.Egr-TNF.11D and doxorubicin produced the greatest reduction in mean tumor volume, reaching a nadir (90% reduction) at day 13 that persisted for the duration of the experiment. A significant difference between the Ad.Egr-TNF.11D alone group and the combination group was detectable on day 16 (p=0.025) and continued until day 23 (p=0.006, FIG. 11A). These results indicate that the combination of Ad.Egr-TNF.11D and doxorubicin overcomes the lack of response to doxorubicin. Systemic toxicity was observed in 30% of mice exposed to doxorubicin. Importantly, these adverse effects were not increased with the combination of Ad.Egr-TNF.11D and doxorubicin.

The effects of Ad.Egr-TNF.11D alone, doxorubicin alone and the combination of both agents were studied in similar experiments performed in PROb xenografts. Tumors with a mean volume of 318.3±18 mm³ (n=40) at day 0 were employed. There was no difference in tumor growth delay at day 27 among the buffer injected control group (mean fractional volume=7.6), the doxorubicin alone group (mean fractional volume=6.9) and the Ad.Egr-TNF.11D alone group (mean fractional volume=6.7). Notably, treatment with Ad.Egr-TNF.11D and doxorubicin produced a significant reduction in mean fractional tumor volume compared with Ad.Egr-TNF.11D alone at day 23 (4.1 versus 5.4; p=0.027). At day 27, tumors in the Ad.Egr-TNF.11D and doxorubicin group exhibited a 4.9-fold increase in fractional tumor volume compared with a 6.7-fold increase in the Ad.Egr-TNF.11D alone group (p=0.015, FIG. 11B). These results suggest that combination treatment with Ad.Egr-TNF.11D and doxorubicin overcomes resistance to both doxorubicin and TNF-α. Toxicity, including weight loss and deaths, was observed in groups receiving doxorubicin alone; however, these effects were not increased with the addition of Ad.Egr-TNF.11D.

### Example 8

### Combined Treatment with AD.EGR-TNF.11D and Doxorubicin Decreases Tumor Microvessel Density

To study the effects of doxorubicin mediated induction of Ad.Egr-TNF.11D on tumor angiogenesis, CD-31 positive tumor vessels were counted on tissue sections from PC-3 tumors. Sample of PC-3 xenografts (day 27) were obtained following treatment with Ad.Egr-TNF.11D and doxorubicin. Microvessels were visualized in paraffin-embedded tissue sections using anti-CD31 immunohistochemistry and a avidin-biotin peroxidase technique (data not shown).

Combined treatment with Ad.Egr-TNF.11D and doxorubicin reduced the number of vessels per high power field (5.35±0.78) compared with the control group (7.89±0.54, p=0.005), the doxorubicin alone group (6.24±0.35, p=0.069) and the Ad.Egr-TNF.11D alone group (6.5±0.43, p=0.057). In the Ad.Egr-TNF.11D and doxorubicin treatment group there were fewer vessels of all diameters and less branching when compared with tumors from the control group, the doxorubicin alone treatment group and the Ad.Egr-TNF.11D alone treatment group. These results indicate that activation of Ad.Egr-TNF.11D enhances treatment with doxorubicin, at least in part, by inhibiting angiogenesis.

These data suggest that the alteration of doxorubicin resistance by the combination of doxorubicin and Ad.Egr-TNF.11D is due in part to the inhibition of tumor angiogenesis. TNF-60 induces the activity and release of angiostatin converting enzymes (Mauceri et al., 2002). In this regard, angiostatin is elevated in the plasma of tumor bearing mice treated with Ad.Egr-TNF.11D (Mauceri et al., 2002). Additionally, it has been reported (Mauceri et al., 2002; Gately et al., 1996) that human tumor cells, including PC-3 cells, produce enzymes capable of converting plasminogen to angiostatin. Angiostatin is reported to be an effective anti-tumor agent when combined with DNA damaging agents through the inhibition of tumor angiogenesis (Mauceri et al., 1998). Taken together, these data suggest that the anti-tumor activity of doxorubicin and Ad.Egr-TNF.11D is mediated by the inhibitory effects of angiostatin and doxorubicin on tumor angiogenesis.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

I. References

The following references, to the extent that they provide exemplary procedural or other details supplementary to those set forth herein, are specifically incorporated herein by reference.

U.S. Pat. No. 4,201,767

U.S. Pat. No. 4,415,732

U.S. Pat. No. 4,458,066

U.S. Pat. No. 4,587,055

U.S. Pat. No. 4,797,368

U.S. Pat. No. 5,139,941

U.S. Pat. No. 5,354,855

U.S. Pat. No. 5,359,046

U.S. Pat. No. 5,759,566

U.S. Pat. No. 5,762,904

U.S. Pat. No. 5,795,715

U.S. Pat. No. 5,889,136

U.S. Pat. No. 5,925,565

U.S. Pat. No. 5,935,819

U.S. Pat. No. 5,994,136

U.S. Pat. No. 6,013,516

U.S. patent application Ser. No. 10/117,442, filed Apr. 5, 2002.

U.S. patent application Ser. No. 60/282,040, filed Apr. 6, 2001.

Abate et al., Science, 249:1157-1161, 1990.

Alexander et al., J. Clin. Oncol., 16:1479-1489, 1998.

Angel, et al., Cell, 55(5):875-885, 1988.

Arap et al., Cancer Res., 55:1351-1354, 1995.

Baichwal and Sugden, In: Gene Transfer, Kucherlapati R, ed., New York, Plenum Press, pp. 117-148, 1986.

Bajorin et al., Proc. Annu. Meet. Am. Soc. Clin. Oncol., 7:A967, 1988.

Bedzyk et al., J. Biol. Chem., 265:18615, 1990

Benvenisty and Neshif, Proc. Natl. Acad. Sci. USA, 83(24):9551-9555, 1986.

Blomer et al., J. Virol., 71(9): 6641-6649, 1997

Bonavida et al., Gynecol. Oncol., 38:333-9, 1990.

Bosher and Labouesse, Nat. Cell. Biol., 2:E31-E36, 2000.

Brenner et al., Nature, 337(6208):661-663, 1989.

Brown et al., J. Immunother., 10:376-378, 1991.

Budd et al., Cancer, 68:1694-1695, 1991.

Burbage et al., Leuk Res., 21(7):681-690, 1997.

Buscher et al., Oncogene, 3(3):301-311 1988.

Bussemakers et al., Cancer Res., 52:2916-2922, 1992.

Caldas et al., Nat. Genet., 8:27-32, 1994.

Cao et al., Mol. Cell Biol., 10(5):1931-9, 1990.

Caplen et al., Gene, 252(1-2):95-105, 2000.

Carbonelli et al. FEMS Microbiol. Lett., 177(1):75-82, 1999.

Carswell et al., Proc. Natl. Acad. Sci. USA, 72:3666-3670, 1975.

Casey et al., Oncogene, 6:1791-1797, 1991.

Cemazar et al., DNA Cell Biol., 22:765-775, 2003.

Chandler et al., Proc. Natl. Acad. Sci. USA, 94(8):3596-3601, 1997.

Chaudhary et al. Proc. Natl. Acad. Sci., 87:9491, 1990

Chen and Okayama, Mol. Cell Biol., 7:2745-2752, 1987.

Cheng et al., Cancer Res., 54:5547-5551, 1994.

Cheung et al., Arch Biochem Biophys, 305(2):563-569, 1993.

Christoforidis et al., Eur. J. Surg. Oncol., 28:875-890, 2002.

Christy et al., Proc. Natl. Acad. Sci. USA, 85(21):7857-7861, 1988.

Chung et al., Cancer Gene Ther., 5:344-349, 1998.

Cocea, Biotechniques, 23:814-816, 1997.

Cotten et al., Proc. Natl. Acad. Sci. USA, 89(13):6094-6098, 1992.

Coupar et al., Gene, 68:1-10, 1988.

Curiel, In: Viruses in Human Gene Therapy, J.-M. H. Vos (Ed.), Carolina Academic Press, Durham, N.C., pp 179-212, 1994.

Datta et al., Proc. Natl. Acad. Sci. USA, 89:10149-10153, 1992.

Datta et al., Proc. Natl. Acad. Sci. USA, 90:2419-2422, 1993.

Demetri et al., J. Clin. Oncol., 7:1545-53, 1989.

Doroshow, Biochem. Biophys. Res. Commun., 135:330-335, 1986.

Duan et al., J. Neurooncol., 52:23-36, 2001.

Dubensky et al., Proc. Natl. Acad. Sci. USA, 81:7529-7533, 1984.

Edelman and Crossin, Annu. Rev. Biochem., 60:155-190, 1991.

Edelman, Annu. Rev. Biochem., 54:135-169, 1985.

Elbashir et al., Genes Dev., 5(2):188-200, 2001.

Elbashir et al., Nature, 411(6836):494-498, 2001.

Fechheimer et al., Proc. Natl. Acad. Sci. USA, 84:8463-8467, 1987.

Fiers, FEBS Letters, 285(2):199-212, 1991.

Fire et al., Nature, 391:806-811, 1998.

Fisch et al., Mol. Cell Biol., 7(10):3490-3502, 1987.

Forster and Symons, Cell, 49:211-220, 1987.

Fraley et al., Proc. Natl. Acad. Sci. USA, 76:3348-3352, 1979.

Friedmann, Science, 244:1275-1281, 1989.

Frixen et al., J. Cell Biol., 113:173-185, 1991.

Gately et al., Cancer Res., 56:4887-4890, 1996.

Gerlach et al., Nature (London), 328:802-805, 1987.

Gessler, Hum. Genet., 86(2):135-8, 1990.

Giancotti and Ruoslahti, Cell, 60:849-859, 1990.

Gilman, Genes Dev., 2(4):394-400, 1988.

Gonen et al., Cancer Immunol. Immunother., 35:388-394, 1992.

Gonzalez-Zulueta et al., Cancer Research, 55(20):4531-4535, 1995.

Gopal, Mol. Cell Biol., 5:1188-1190, 1985.

Graham and Van Der Eb, Virology, 52:456-467, 1973.

Grishok et al., Science, 287:2494-2497, 2000.

Grunhaus and Horwitz, Seminar in Virology, 3:237-252, 1992.

Gupta et al., Ann. Surg. Oncol., 9:500-504, 2002.

Halazonetis et al., Cell, 55(5):917-924, 1988.

Hall, Radiobiology for the Radiologist, Harper and Row, 1988.

Hall, Radiobiology for the Radiologist, Harper and Row, 1994.

Hallahan et al., Cancer J. Sci. Am., 1:204, 1995.

Hallahan et al., Nat. Med., 1:786-791, 1995.

Hanna et al., Proc. Of ASCO, Vol. 21, 2002.

Harland and Weintraub, J. Cell Biol., 101:1094-1099, 1985.

Harlow and Lane, Antibodies: A Laboratory manual, Cold Spring Harbor Laboratory, 1988.

Havell et al., J Exp Med, 167:1067-85, 1988.

Herman et al., Cancer Research, 55(20):4525-4530, 1995.

Hill et al., Br. J. Surg., 80:995-997, 1993.

Hollstein et al., Science, 253:49-53, 1991.

Homma et al., Proc. Natl. Acad. Sci. USA, 83(19):7316-7319, 1986.

Horwich et al. J. Virol., 64:642-650, 1990.

Houben, Int. J. Radiat. Biol. Relat. Stud. Phys. Chem. Med., 20:373-389, 1971.

Hussussian et al., Nature Genetics, 15-21, 1994.

Johnson and Stevenson, In: Cancer.Principles and Practice of Oncology (ed. DeVita, V. T., Hellman and Rosenberg, ) 376-88, 2001.

Joyce, Nature, 338:217-244, 1989.

Kamb et al., Science, 2674:436-440, 1994.

Kaneda et al., Science, 243:375-378, 1989.

Kartalou and Essigmann, Mutat Res, 478:23-43, 2001.

Kelleher and Vos, Biotechniques, 17(6): 1110-1117, 1994.

Ketting et al., Cell, 99:133-141, 1999.

Kim and Cook, Proc. Natl. Acad. Sci. USA, 84:8788-8792, 1987.

Klein et al., Nature, 327:70, 1987.

Konishi et al., J. Med. Sci., 48:79-89, 1999.

Kubota, Anti-Cancer Drugs, 2:531-541, 1991.

Kucuk et al., Am. J. Clin. Oncol., 23:371-5, 2000.

Kuppen et al., Br. J. Cancer, 75:1497-1500, 1997.

Kurihara et al., J. Clin. Invest., 106:763-771, 2000.

Laughlin et al., J. Virol., 60(2):515-524, 1986.

Lebkowski et al., Mol. Cell Biol., 8(10):3988-3996, 1988.

Lejeune et al., Circ. Shock, 43:191-197, 1994.

Lemaire et al., Proc. Natl. Acad. Sci. USA, 85(13):4691-4695, 1988.

Levenson et al., Human Gene Therapy, 9:1233-1236, 1998.

Li et al., Exp. Eye Res., 59:179-190, 1994.

Lidor et al., Am. J. Obstet. Gynecol.;177(3):579-585, 1997.

Lienard et al., Melanoma Res., 4(1):21-26, 1994.

Lin and Avery, Nature, 402:128-129, 1999.

Lin and Guidotti, J. Biol. Chem., 264:14408-14414, 1989.

Macejak and Sarnow, Nature, 353:90-94, 1991.

Mann et al., Cell, 33:153-159, 1983.

Massuda et al., Proc. Natl. Acad. Sci. USA, 94(26):14701-14706, 1997.

Matsura et al., Brit. J. Cancer, 66:1122-1130, 1992.

Mauceri et al., Cancer Res., 56:4311-4314, 1996.

Mauceri et al., Int J Cancer, 97:410-5, 2002.

Mauceri et al., Nature, 394:287-291, 1998.

Mauceri et al., Radiat. Oncol. Investig., 5:220-226, 1997.

McLaughlin et al., J. Virol., 62(6):1963-1973, 1988.

Merlo et al., Nat Med., (7):633-4, 1995.

Michel and Westhof, J. Mol. Biol., 216:585-610, 1990.

Milbrandt, Science, 238(4828):797-799, 1987.

Miller et al., Am. J. Clin. Oncol., 15(3):216-221, 1992.

Mitchell et al., Ann. NY Acad. Sci., 690:153-166, 1993.

Mitchell et al., J. Clin. Oncol, 8(5):856-869, 1990.

Mitchell et al., Science, 245(4916):371-378, 1989

Mittelman et al., Invest. New Drugs, 10:193-190, 1992.

Mori et al., Cancer Res., 54:3396-3397, 1994.

Montgomery et al., Proc. Natl. Acad. Sci. USA, 95:155-2-15507, 1998.

Mundt et al., In: 14^{th} EORTC-NCI-AACR Symposium on Molecular Targets and Cancer Therapeutics, Frankfurt, Germany, 2002.

Muzyczka, Curr. Top. Microbiol. Immunol., 158:97-129, 1992.

Myers, EPO 0273085.

Nakabeppa et al., Cell, 55(5):907-915, 1988.

Nakamoto et al., Anticancer Res, 20:4087-96, 2000.

Naldini et al., Science, 272(5259):195, 1996

Nawroth and Stem, J. Exp. Med., 163:740-745, 1986.

Nicolas and Rubenstein, In: Vectors: A survey of molecular cloning vectors and their uses, Rodriguez and Denhardt (eds.), Stoneham: Butterworth, pp. 493-513, 1988.

Nicolau and Sene, Biochim. Biophys. Acta, 721:185-190, 1982.

Nicolau et al., Methods Enzymol., 149:157-176, 1987.

Nobri et al., Nature, 368:753-756, 1995.

Nose et al., Eur. J. Biochem., 201:99-106, 1991.

Obrador et al., Curr. Pharm. Biotechnol., 2:119-30, 2001.

Obrink, BioEssays, 13:227-233, 1991.

Odin and Obrink, Exp. Cell Res., 171:1-15, 1987.

Okamoto et al., Proc. Natl. Acad. Sci. USA, 91:11045-11049, 1994.

Old, Science, 230:630-2, 1985.

Orlow et al., Cancer Res., 54:2848-2851, 1994.

Park et al., J. Clin. Invest., 110:403-410, 2002.

Paskind et al., Virology, 67:242-248, 1975.

PCT Appl. WO 00/44914

PCT Appl. WO 01/36646

PCT Appl. WO 01/68836

PCT Appl. WO 99/32619

Pelletier and Sonenberg, Nature, 334:320-325, 1988.

Potter et al., Proc. Natl. Acad. Sci. USA, 81:7161-7165, 1984.

Prywes et al., Proc. Natl. Acad. Sci. USA, 85(19):7206-7210, 1988.

Quinones et al., Life Sci., 72:2975-2992, 2003.

Qureshi et al., J. Biol. Chem., 266(31):20594-7, 1991.

Ravindranath and Morton, Intern. Rev. Immunol., 7: 303-329, 1991.

Reinhold-Hurek and Shub, Nature, 357:173-176, 1992.

Remington's Pharmaceutical Sciences, 15th ed., 1035-1038 and 1570-1580.

Ridgeway, In: Vectors: A survey of molecular cloning vectors and their uses, Rodriguez and Denhardt (Eds.), Stoneham:Butterworth, 467-492, 1988.

Rippe et al., Mol. Cell Biol., 10:689-695, 1990.

Robaye et al., Am. J. Pathol., 138:447-53, 1991.

Roux et al., Proc. Natl Acad. Sci. USA, 86:9079-9083, 1989.

Ruff and Gifford, Infect. Immun., 31:380-385, 1981.

Sambrook et al., In: Molecular cloning, Cold Spring Harbor Laboratory Press, Cold Spring Harbor, N.Y., 2000.

Sarver et al., Science, 247:1222-1225, 1990.

Scanlon et al., Proc. Natl. Acad. Sci. USA, 88:10591-10595, 1991.

Senturker et al., Arch. Biochem. Biophysics, 397:262-272, 2002.

Senzer et al., In: Gene: A Phase I Study in Patients with Solid Tumors, J. Clin. Oncol., 2004.

Serrano et al., Nature, 366:704-707, 1993.

Serrano et al., Science, 267:249-252, 1995.

Sharma et al., Human Gene Therapy, 12:1109-1131, 2001a.

Sharma et al., In: American Society of Therapeutic Radiation and Oncology, 43^{rd} Annual Meeting, Abstract 187, 2001b.

Sharp and Zamore, Science, 287:2431-2433, 2000.

Sharp, Genes Dev., 13:139-141, 1999.

Sheng et al., Nature, 337(6208):661, 1988.

Sherman et al., Proc. Natl. Acad. Sci. USA, 87(15):5663-5666, 1990.

Sinha et al., Biochim. Biophys. ACTA, 1270:12-18, 1995.

Slungaard et al., J. Exp. Med., 171:2025-41, 1990.

Smets, Anti-Cancer Drugs, 5:3-9, 1994.

Smith and Rutledge, Natl. Cancer Inst. Monogr., 42:169-172, 1975.

Sodhi and Gupta, Int. J. Immunopharmacol., 8:709-714, 1986.

Spriggs et al., Ciba Found. Symp., 131:206-227, 1987.

Spriggs et al., J. Natl. Cancer Inst., 80:1039-44, 1988.

Staba et al., Gene Therapy, 5:293-300, 1998.

Stumpo et al., J. Biol. Chem., 263(4):1611-1614, 1988.

Sukhatme et al. Cell, 53(1):37-43, 1988.

Sukhatme et al., Cell, 8;53(1):37-43 1988.

Sulkowska et al., Exp. Toxicol. Pathol., 50:209-220, 1998.

Tabara et al., Cell, 99:123-132, 1999.

Takahashi et al., Cancer Res., 52:734-736, 1992

Tartaglia et al., Cell, 74:845-53, 1993.

Teicher et al., In Vivo, 11:453-461, 1997.

Temin, In: Gene Transfer, Kucherlapati (ed.), New York: Plenum Press, pp. 149-188, 1986.

Thom et al., J. Clin. Oncol., 13:264-73, 1995.

Tratschin et al., Mol. Cell. Biol., 4:2072-2081, 1984.

Treisman, Cell, 46(4):567-174, 1986.

Tur-Kaspa et al., Mol. Cell Biol., 6:716-718, 1986.

Ueta et al., Jpn. J. Cancer Res., 90:555-564, 1999.

Umbas et al., Cancer Res., 52:5104-5109, 1992.

van Brussel et al., Eur. J. Cancer, 35:664-671, 1999.

van der Donk et al., Biochemistry, 37:6419-6426, 1998.

Varbiro et al., Free Radic. Biol. Med., 31:548-558, 2001.

Vilcek et al., J. Exp. Med., 163:632-643, 1986.

Wallach, J. Immunol., 132:2464-2469, 1984.

Watanabe et al., Cancer Res., 48:2179-83, 1988.

Weinberg, Science, 254:1138-1145, 1991.

Wiedenmann et al., J. Cancer Res. Clin. Oncol., 115:189-192, 1989.

Wincott et al., Nucleic Acids Res., 23(14):2677-2684, 1995.

Wu and Wu, Adv. Drug Delivery Rev., 12:159-167, 1993.

Wu and Wu, Biochem., 27:887-892, 1988.

Wu and Wu, J. Biol. Chem., 262:4429-4432, 1987.

Yang et al., Proc. Natl. Acad Sci. USA, 87:9568-9572, 1990.

Young et al., N Engl J Med. 7:299(23):1261-1266, 1978.

Zafaruuah et al., Cell Mol. Life Sci., 60:6020, 2003.

Zufferey et al., Biotechnol, 15(9):871-875, 1997.

What is claimed is:

1. A method of inhibiting a hyperproliferative cell comprising contacting said cell with:
(a) an expression construct, said expression construct comprising a chemotherapeutic responsive promoter, said promoter operably linked to a nucleic acid encoding tumor therapeutic gene; and
(b) a chemotherapeutic agent selected from doxorubicin, cyclophosphamide, 5-fluorouracil, taxol or gemcitabine.

2. The method of claim 1, wherein said promoter is the Egr-1 promoter.

3. The method of claim 1, wherein said promoter is the c-jun promoter.

4. The method of claim 1, wherein said promoter is the c-fos promoter.

5. The method of claim 1, wherein said chemotherapeutic agent is doxorubicin.

6. The method of claim 1, wherein said hyperproliferative cell is a cancer cell or a metastatic cancer cell.

7. The method of claim 6, wherein said tumor therapeutic gene is TNF-α.

8. The method of claim 5, wherein said cancer cell is a multi-drug resistant cancer cell.

9. The method of claim 6, wherein said cancer cell is a breast cancer, an ovarian cancer, a pancreatic cancer, a prostate cancer, a colon cancer, a bladder cancer, a lung cancer, a liver cancer, a stomach cancer, a testicular cancer, an uterine cancer, a brain cancer, a lymphatic cancer, a skin cancer, a bone cancer, a kidney cancer, a rectal cancer, or a sarcoma.

10. The method of claim 1, wherein said hyperproliferative cell is located in a mammal.

11. The method of claim 10, wherein said hyperproliferative cell is a recurrent cancer cell.

12. The method of claim 10, wherein said mammal is a human.

13. The method of claim 1, wherein said chemotherapeutic agent is cyclophosphamide.

14. The method of claim 1, wherein said chemotherapuetic agent is 5-fluorouracil.

15. The method of claim 1, wherein said chemotherapuetic agent is taxol.

16. The method of claim 1, wherein said chemotherapuetic agent is gemcitabine.

17. The method of claim 1, wherein two or more of doxorubicin, cyclophosphamide, 5-fluorouracil, taxol or gemcitabine are contacted with said cell.

18. The method of claim 2, wherein said expression construct is a viral expression construct.

19. The method of claim 3, wherein said expression construct is a non-viral expression construct.

20. The method of claim 18, wherein said viral vector is an adenoviral vector, adeno-associated viral vector, retroviral vector, lentiviral vector, herpesviral vector, papilloma viral vector, or hepatitis B viral vector.

21. The method of claim 19, wherein said non-viral vector is comprised in a liposome.

22. The method of claim 1, wherein said tumor therapeutic gene is delivered to a cell at the same time as said chemotherapeutic agent.

23. The method of claim 10, wherein inhibiting comprises inhibiting metastasis of said cancer cell.

24. The method of claim 10, wherein inhibiting comprises reducing tumor burden in said mammal.

25. The method of claim 10, wherein inhibiting compries inducing tumor regression in said mammal.

26. The method of claim 1, wherein inhibiting comprises killing said hyperproliferative cell.

27. The method of claim 1, wherein inhibiting comprises inducing apoptosis in said hyperproliferative cell.

28. The method of claim 10, wherein said tumor therapeutic gene is administered more than once.

29. The method of claim 10, wherein said chemotherapeutic agent is administered more than once.

30. The method of claim 10, wherein said tumor therapeutic gene is administered intratumorally, intramuscullarly, intravenously or intraaterially.

31. The method of claim 10, wherein said chemotherapuetic agent is administered intratumorally, intramuscularly, intravenously or intraaterially.

32. The method of claim 1, wherein said tumor therapeutic gene is delivered to a cell after said chemotherapeutic agent.

33. The method of claim 1, wherein said tumor therapeutic gene is delivered to a cell before said chemotherapeutic agent.

34. The method of claim 10, further comprising providing said mammal with an adjunct cancer therapy.

35. The method of claim 34, wherein the cancer adjunct therapy is a second chemotherapy, a radiotherapy, an immunotherapy, a hormonal therapy, or a gene therapy.
